(19) United States
(12) Patent Application Publication
(10) Pub. No.: US 2025/0359792 A1
(43) Pub. Date: Nov. 27, 2025

(54) DELIRIUM CLASSIFICATION COMPUTING SYSTEM

(71) Applicant: Lucidify, Inc., San Francisco, CA (US)

(72) Inventor: Divya Chander, San Francisco, CA (US)

(21) Appl. No.: 19/216,563

(22) Filed: May 22, 2025

Related U.S. Application Data
(60) Provisional application No. 63/651,339, filed on May 23, 2024.

(51) Int. Cl. A61B 5/16; A61B 5/369; G16H 50/20

(52) U.S. Cl. CPC A61B 5/165; A61B 5/369; G16H 50/20

(57) ABSTRACT

A delirium detection computing system includes a sensor system comprising a one or more electroencephalography (EEG) electrodes configured to generate a plurality of EEG signals. The system also includes processing circuitry and a non-volatile memory storing executable instructions that, in response to execution by the processing circuitry, cause the processing circuitry to receive the plurality of EEG signals, preprocess the received plurality of EEG signals to generate preprocessed EEG signals, extract features to generate EEG representations based on the preprocessed EEG signals, execute a delirium classifier to generate a classification indicating whether or not delirium is likely, based on the generated EEG representations, and generate and output one or more notifications based on the generated classification.

### CROSS REFERENCE TO RELATED APPLICATIONS

This application claims priority to U.S. Provisional Patent Application Ser. No. 63/651,339, filed May 23, 2024, the entirety of which is hereby incorporated herein by reference for all purposes.

### BACKGROUND

The brain can experience a number of different brain states that impact a patient's health. For example, delirium, a severe disturbance in mental abilities resulting in confused thinking and reduced awareness of the environment, presents a significant and urgent medical need. Its detection and early diagnosis are a particularly acute challenge, one that has been intensified by the COVID-19 crisis. The pandemic has not only increased the incidence of delirium due to higher hospitalization and ICU admissions, but also highlighted the lack of remote, continuous, objective diagnostics for this critical failure of brain and cognitive function. Other brain states can similarly affect patient medical needs in significant ways.

The early detection of brain states such as delirium is an imperative for timely intervention and treatment so that a rapid deterioration in the patient's condition can be averted. Timely treatment can include addressing the underlying causes (such as infections, organ dysfunction, medication adjustments, or dehydration), as well as implementing non-pharmacological interventions to manage symptoms. Further, undetected or late-detected delirium is associated with various complications, including falls, prolonged hospitalization, increased risk of developing dementia, and even higher mortality rates. Early detection reduces these risks by allowing healthcare providers to implement preventative measures and tailored care plans.

Unfortunately, the current reliance on clinician screening for the detection of brain states such as delirium is fraught with limitations, including frequent oversight by healthcare providers. The gold standard for delirium detection is a detailed clinical exam, based on the Diagnostic and Statistical Manual of Mental Disorders, 5^{th} Edition Text Revision (DSM-5-TR), or a proxy examination like the Confusion Assessment Method for the Intensive Care Unit (CAM-ICU), often a questionnaire that functions as a clinical screening tool, much as the Mini-Mental Status Exam (MMSE) does. A positive screen brings a neurologist or intensivist to confirm diagnosis. Other subjective diagnostics are being developed that are easier to administer. But no objective, technological measure for the early detection of delirium has been developed. Structural neuroimaging technologies like functional magnetic resonance imaging (fMRI) are large, cumbersome, and static snapshots, and would require the transport of patients that are critically ill, making them ill-suited to continuous monitoring.

In contrast, electroencephalography (EEG) offers a more practical approach for ongoing monitoring of brain states. However, its application has been challenging, especially in terms of continuous recording in settings beyond specialized laboratories, requiring a large amount of time for application of the electrode arrays and specialized MD interpretation, making them a scarce resource. Full EEG arrays (i.e. the 10-20 system) are also not comfortable for patients to wear continually, especially to lie on in the supine position.

### SUMMARY

To address the above issues, a delirium classification computing system is provided, comprising a sensor system comprising one or more electroencephalography (EEG) electrodes configured to generate a plurality of EEG signals. The system also includes processing circuitry and a non-volatile memory storing executable instructions that, in response to execution by the processing circuitry, cause the processing circuitry to receive the plurality of EEG signals, preprocess the received plurality of EEG signals to generate preprocessed EEG signals, extract features to generate EEG representations based on the preprocessed EEG signals, execute a delirium classifier to generate a classification indicating whether or not delirium is likely, based on the generated EEG representations, and generate and output one or more notifications based on the generated classification.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter. Furthermore, the claimed subject matter is not limited to implementations that solve any or all disadvantages noted in any part of this disclosure.

### BRIEF DESCRIPTION OF THE DRAWINGS

FIG. 1A shows an illustration depicting a real-time delirium detection system according to a first example embodiment of the subject disclosure.

FIG. 1B shows an illustration depicting a real-time delirium detection system according to a second example embodiment of the subject disclosure.

FIG. 1C shows an illustrative example of the health data that may be inputted and processed by the delirium classifier of FIG. 1B.

FIG. 1D shows an illustration of the visual and/or audio notifications that may be outputted by the output device of the first or second example embodiments of the subject disclosure.

FIG. 2A shows a frontal view illustration of exemplary scalp placements of one or more EEG electrodes of the sensor system of the real-time delirium detection system according to FIG. 1A or 1B.

FIG. 2B shows a left side view illustration of exemplary scalp placements of one or more EEG electrodes of the sensor system of the real-time delirium detection system according to FIG. 1A or 1B.

FIG. 2C shows a right side view illustration of exemplary scalp placements of one or more EEG electrodes of the sensor system of the real-time delirium detection system according to FIG. 1A or 1B.

FIG. 3A shows an illustration of the EEG representations of FIG. 1A or 1B as graphs which represent the power spectra of the EEG signals for each frequency.

FIGS. 3B-F show illustrations of the EEG representations of FIG. 1A or 1B as power ratios dividing the summed absolute power of the EEG signals in all frequencies in one frequency range by the summed absolute power of the EEG signals in all frequencies in another frequency range.

FIG. 4A shows an illustration of the EEG representations of FIG. 1A or 1B as attractors that are generated from time-delayed embeddings observed in three-dimensional state space which are created from preprocessed EEG signals.

FIG. 4B shows an illustration of the EEG representations of FIG. 1A or 1B as an entropy-based measure capturing the nonlinear, complex, and irregular dynamics of EEG signals over time.

FIGS. 5A-B show illustrations of exemplary scalp placements of a pair of EEG electrodes of the sensor system of the real-time delirium detection system according to FIG. 1A or 1B.

FIG. 6A shows a schematic representation of a pair of EEG electrode positions which may be used to measure a degree of synchrony for determining a non-delirium state or a delirium state.

FIG. 6B shows an illustration of the EEG representations of FIG. 1A or 1B as graphs indicating a degree of synchrony or connectivity between two or more EEG electrodes over time.

FIG. 6C shows illustrations of a summary of the EEG representations of FIG. 1A or 1B as a graph indicating a degree of functional connectivity between two or more EEG electrodes across a plurality of frequency ranges.

FIG. 6D shows illustrations of the EEG representations of FIG. 1A or 1B as bar graphs indicating changes in frontal functional connectivity between two or more EEG electrodes across a plurality of frequency ranges.

FIG. 6E shows an illustration of the EEG representations of FIG. 1A or 1B as a graph indicating changes in a degree of frontal to frontotemporal connectivity between two EEG electrodes placed at the Fp1 and F7 positions, respectively, across a plurality of frequency ranges.

FIG. 6F shows an illustration of the EEG representations of FIG. 1A or 1B as a graph indicating changes in a degree of frontal to central connectivity between two EEG electrodes placed at the Fp1 and Fpz positions or the Fp1 and AFz positions, respectively, across a plurality of frequency ranges.

FIG. 6G shows an illustration of the EEG representations of FIG. 1A or 1B as a graph indicating changes in a degree of central to frontal connectivity between two EEG electrodes placed at the Fp1 and Fpz positions or the Fp1 and AFz positions, respectively, across a plurality of frequency ranges.

FIG. 7 shows a flow chart of a first method for real-time delirium detection which may be implemented on the delirium detection computing system illustrated in FIG. 1A or 1B.

FIG. 8 shows a flow chart of a second method for real-time delirium detection which may be implemented on the delirium detection computing system illustrated in FIG. 1A or 1B.

FIG. 9 shows a flow chart of a third method for real-time delirium detection which may be implemented on the delirium detection computing system illustrated in FIG. 1A or 1B.

FIG. 10 shows a flow chart of a fourth method for real-time delirium detection which may be implemented on the delirium detection computing system illustrated in FIG. 1A or 1B.

FIG. 11 shows a flow chart of a fifth method for real-time delirium detection which may be implemented on the delirium detection computing system illustrated in FIG. 1A or 1B.

FIG. 12 shows a flow chart of a sixth method for real-time delirium detection which may be implemented on the delirium detection computing system illustrated in FIG. 1A or 1B.

FIG. 13 shows a schematic view of an example computing environment that can be used according to the systems and methods described herein.

### DETAILED DESCRIPTION

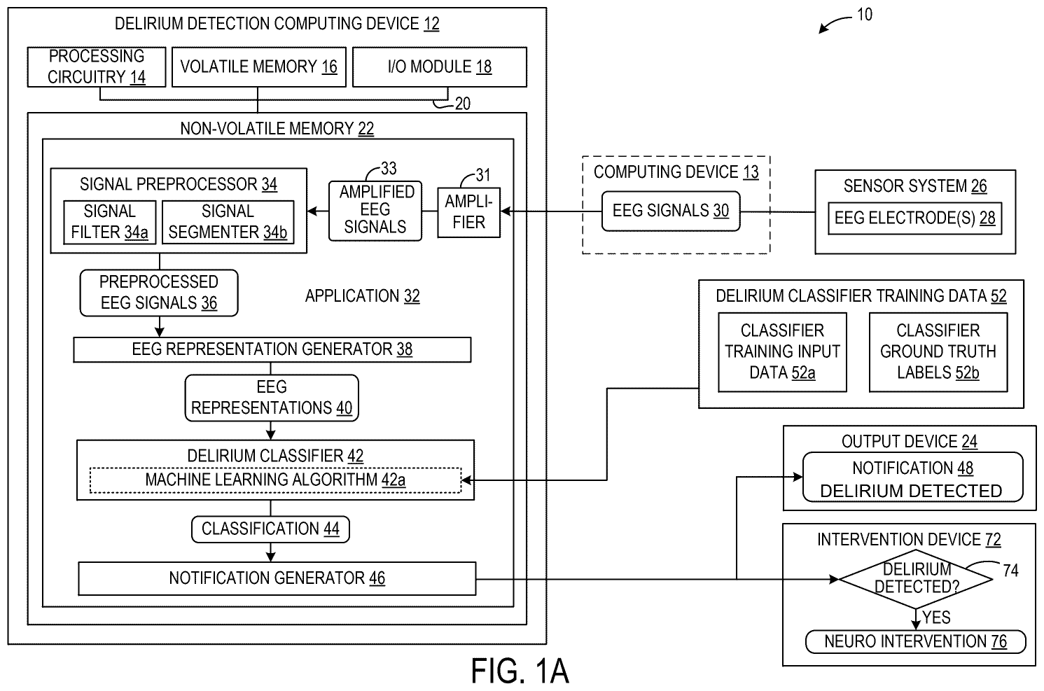

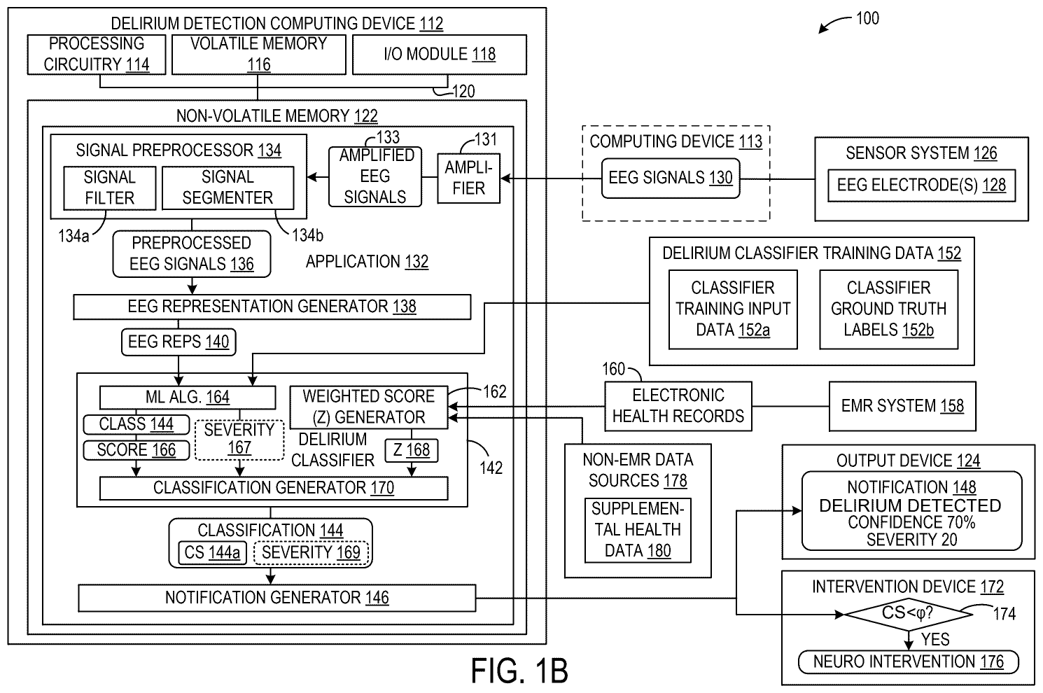

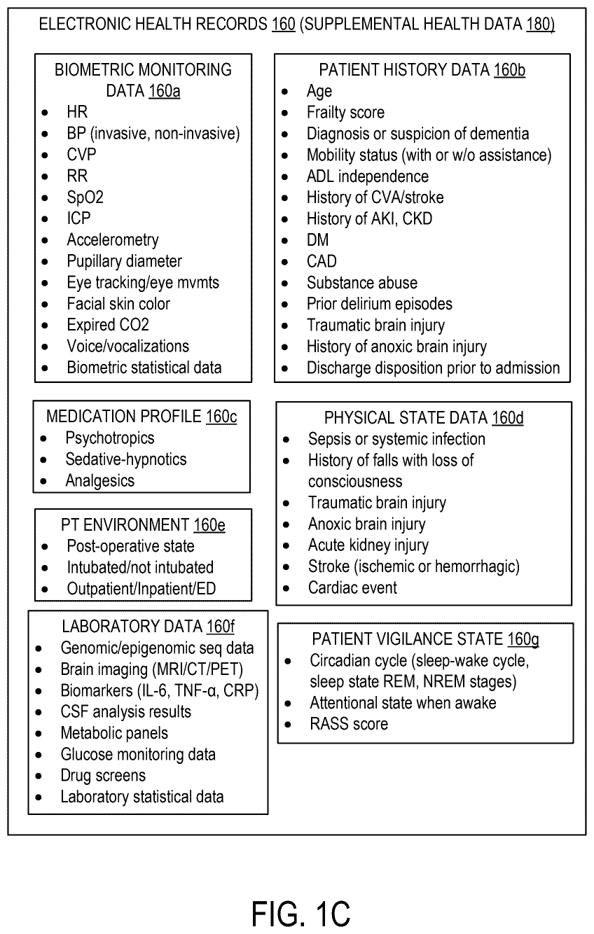

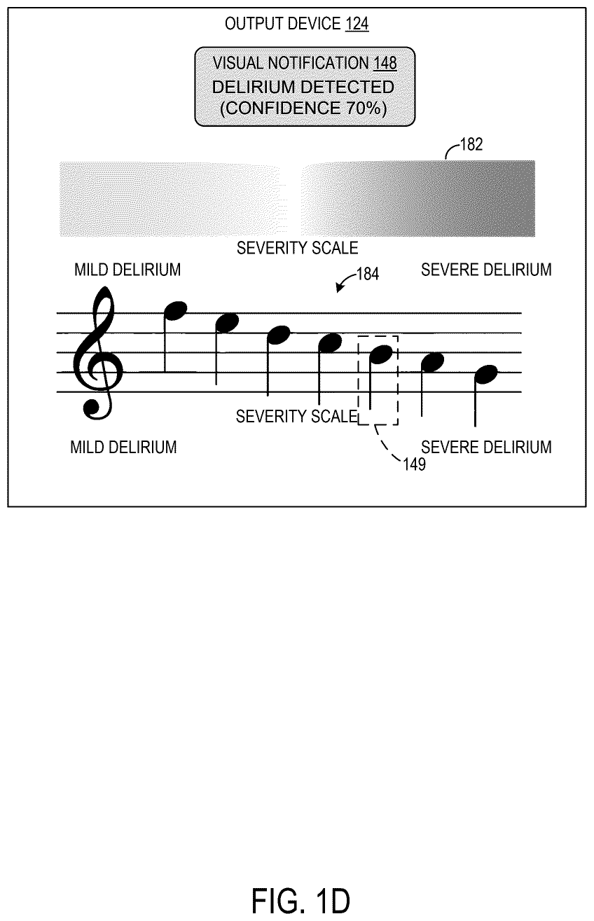

To address the issues described above, FIG. 1A illustrates a first exemplary real-time delirium classification system 10, which can be used to screen patients for a likelihood of delirium based on brain signals. The real-time delirium classification system 10 comprises a delirium classification computing device 12 with a sensor system 26 including one or more EEG electrodes 28 configured to generate EEG signals 30. The computing device 12 comprises processing circuitry 14, volatile memory 16, input/output module 18, and non-volatile memory 22 storing an application 32. It will be appreciated that the number of EEG electrodes 28 is less than conventional configurations with more than 20 EEG electrodes. The number of EEG electrodes 28 is preferably 10 or less, more preferably 4 or less, and still more preferably one or two. In configurations where one or two electrodes are used, the EEG signals 30 may be derived by measuring voltage differences between one electrode and a reference or ground electrode, or by measuring voltage differences between two active electrodes 28.

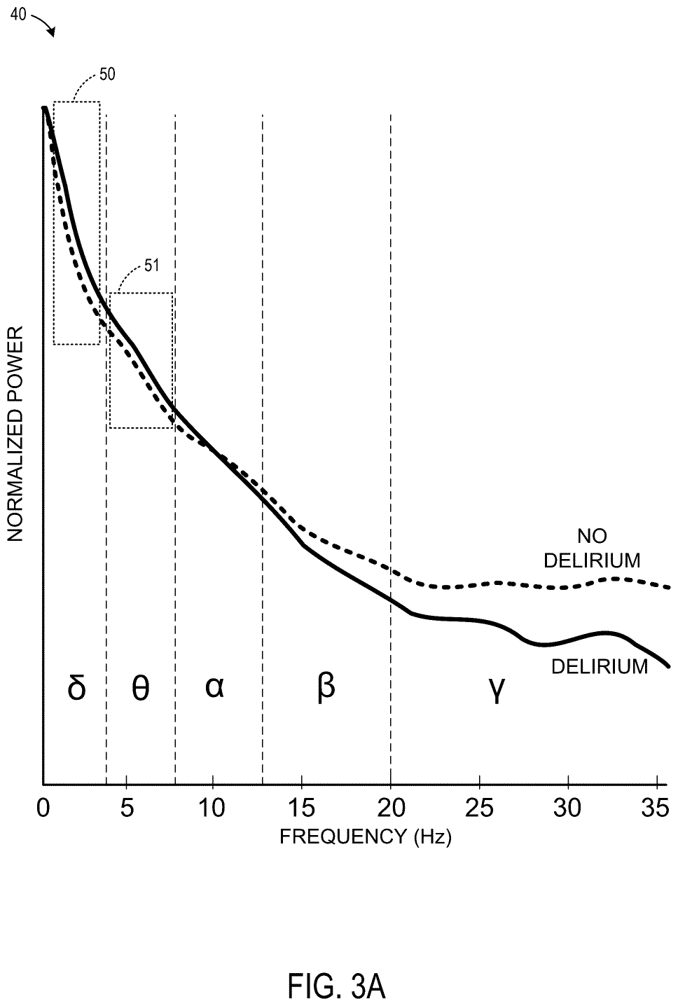

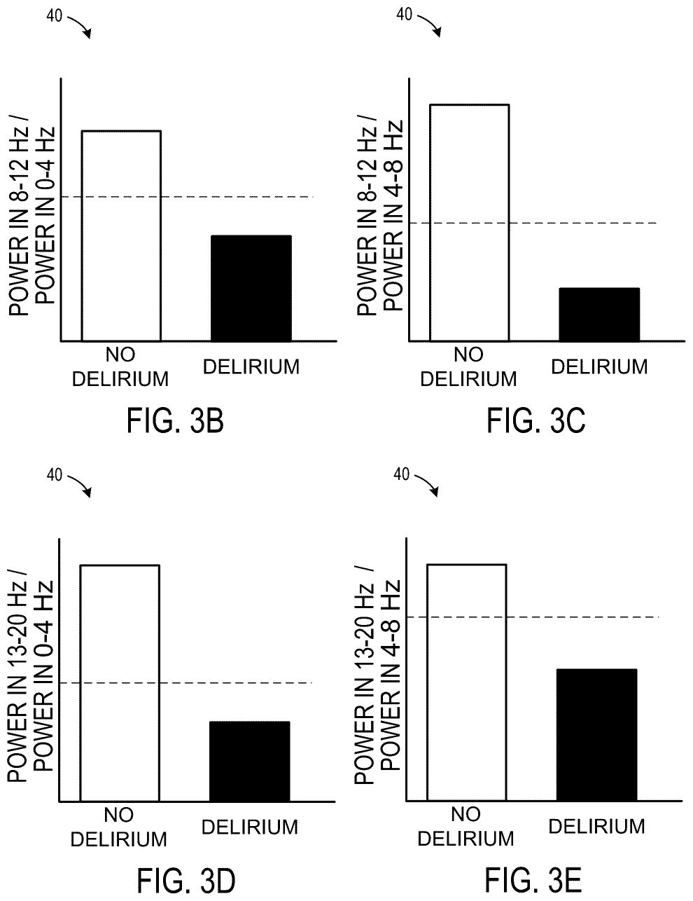

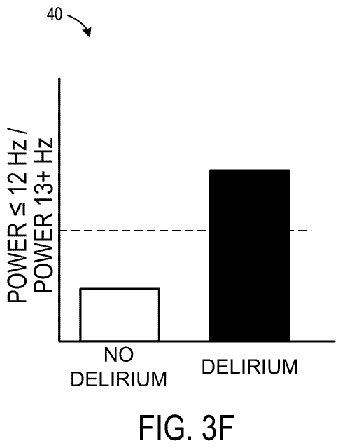

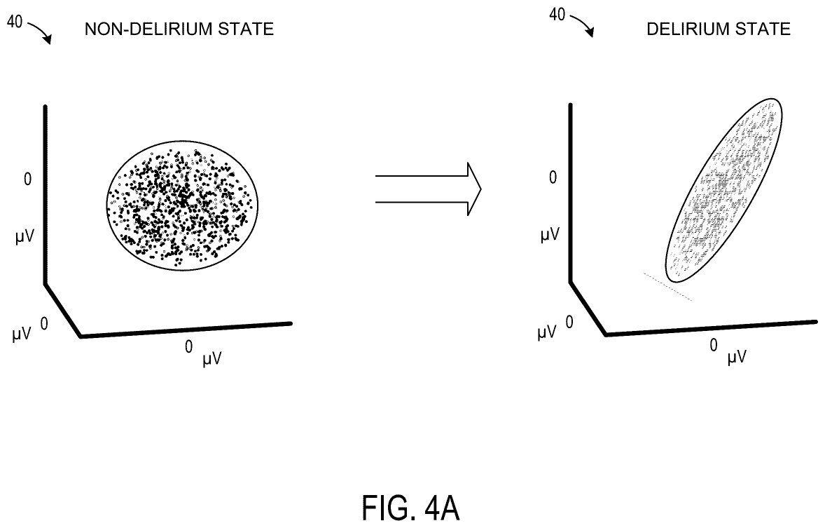

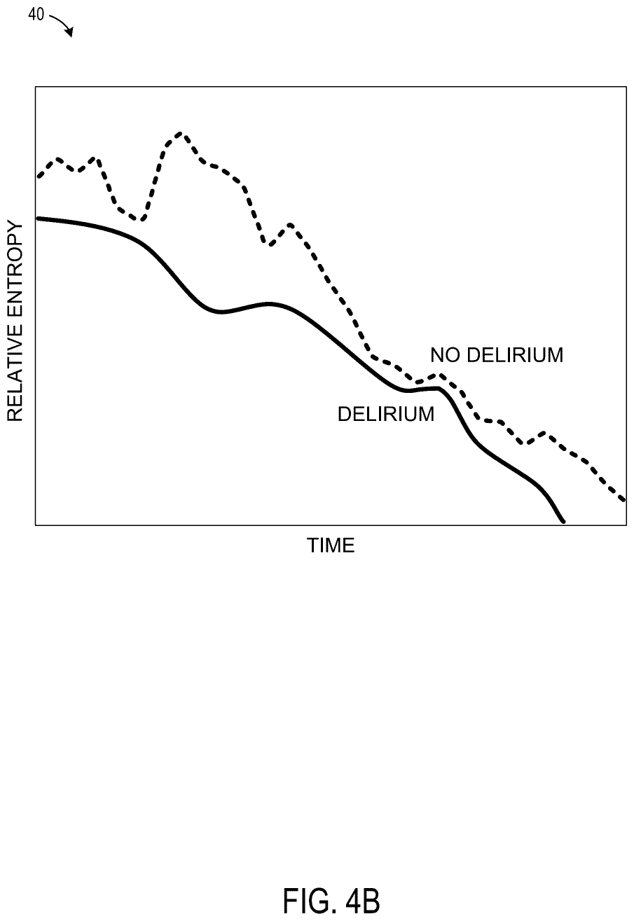

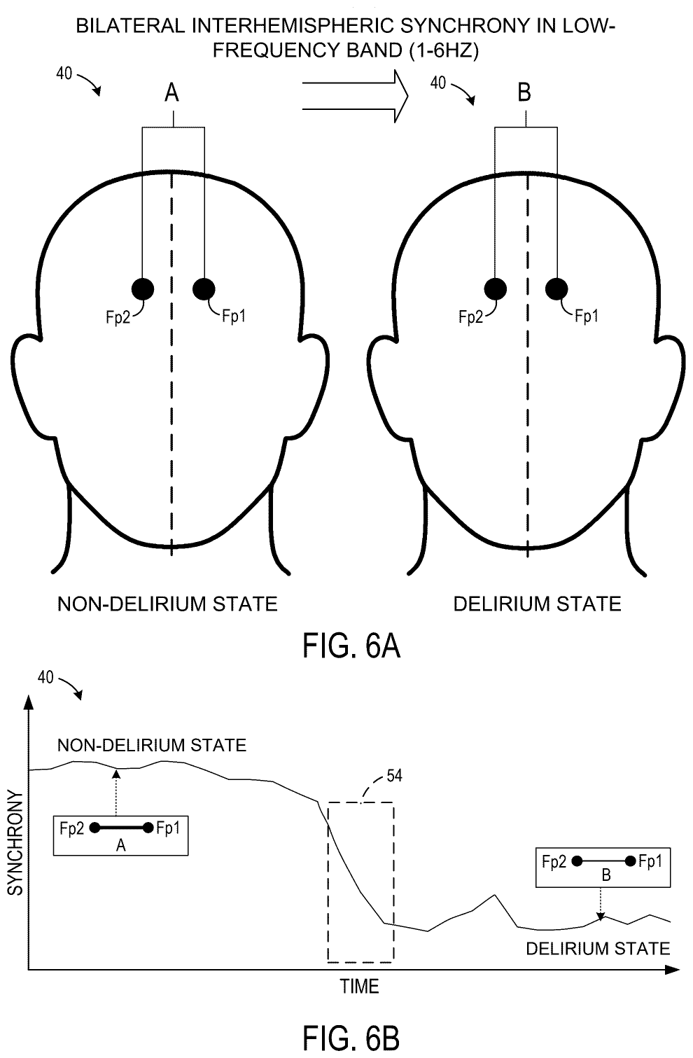

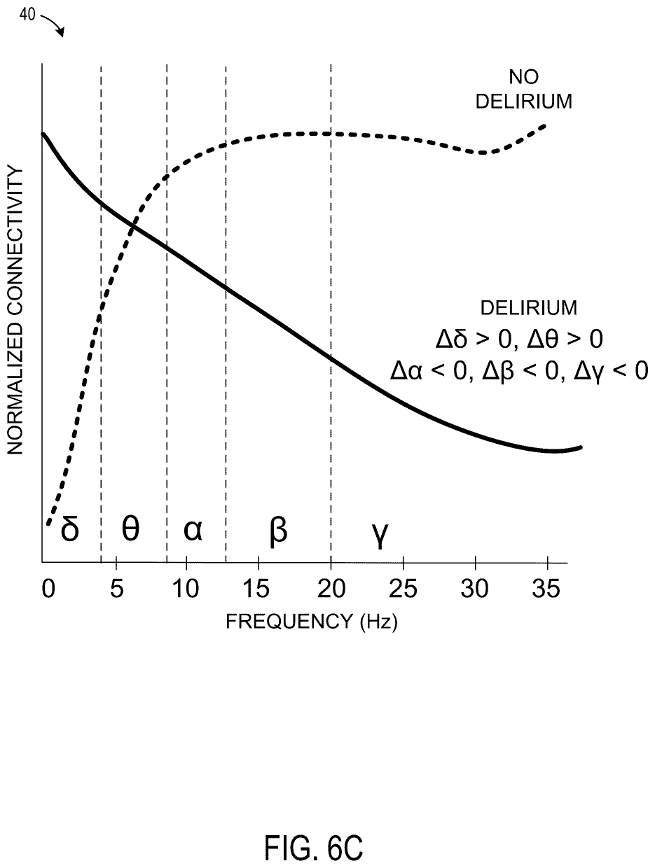

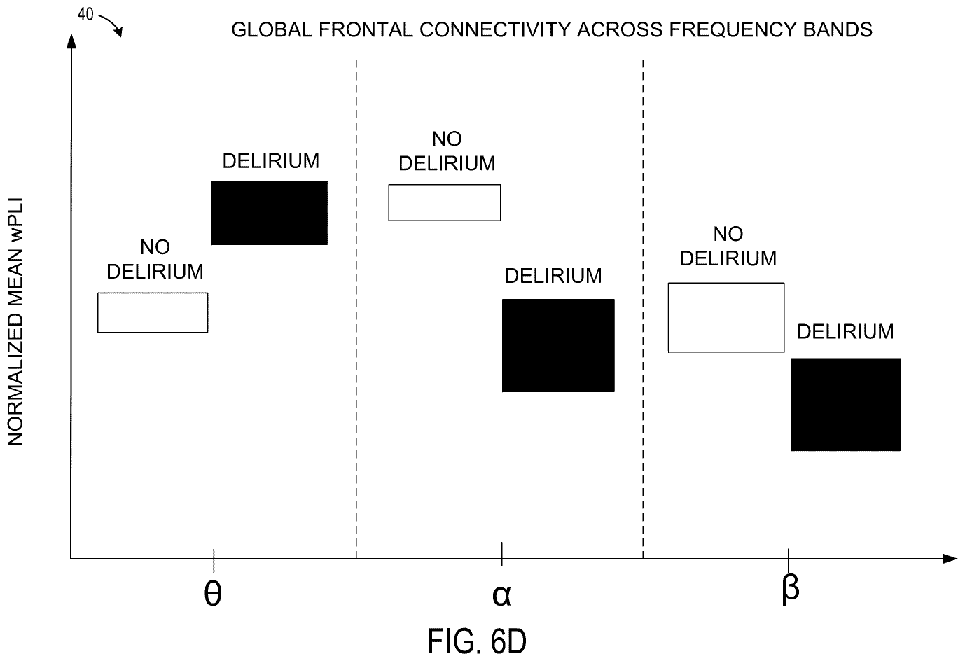

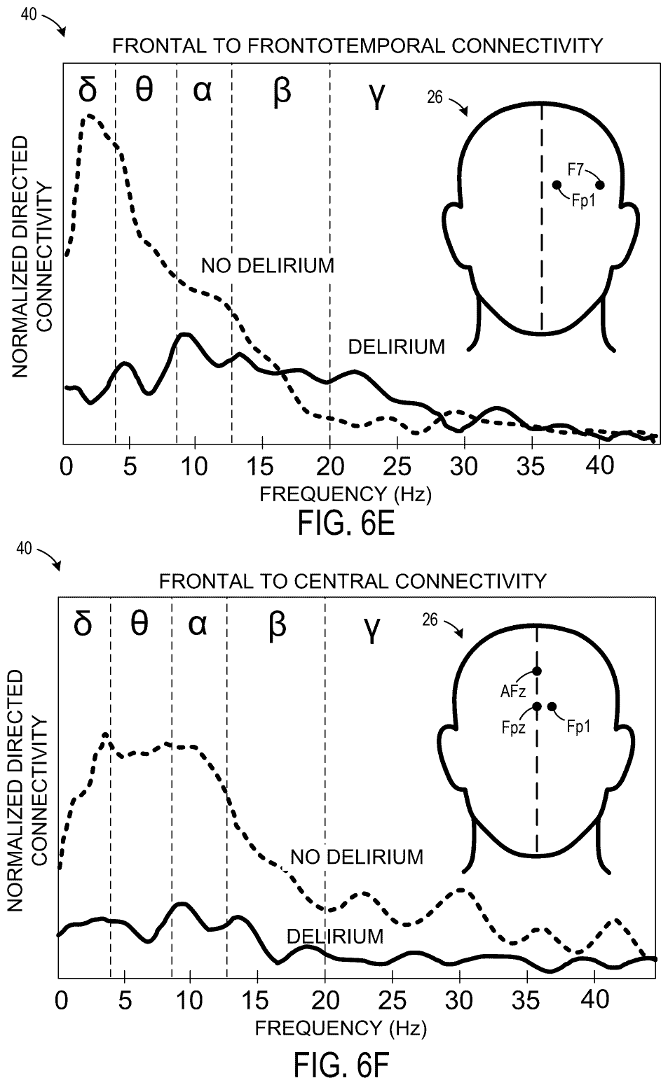

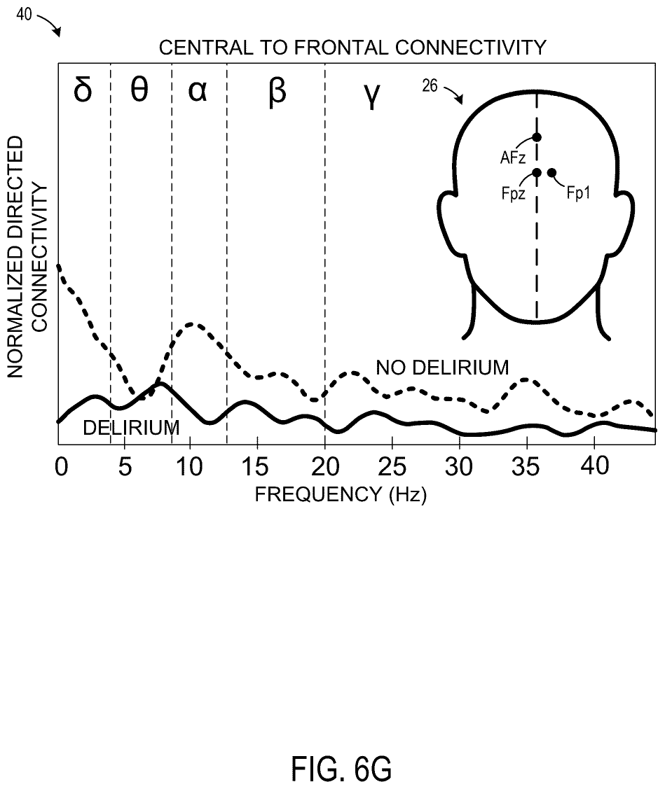

The application 32 causes the processing circuitry 14 to execute an amplifier 31 to amplify the EEG signals to generate amplified EEG signals 33, a signal preprocessor 34 configured to preprocess the amplified EEG signals 33, an EEG representation generator 38 configured to generate an EEG representation 40 based on the preprocessed EEG signals 36, a delirium classifier 42 configured to generate a delirium classification 44 based on the EEG representations 40, and a notification generator 46 configured to generate one or more notifications 48 based on the delirium classification 44. The amplifier 31, the signal preprocessor 34, the EEG representation generator 38, and/or the delirium classifier 42 may be instantiated in one computing device 12, or alternatively instantiated in a plurality of computing devices (in computing device 12 and computing device 13, for example). Computing device 13 may be configured as a gateway device, smartphone, or a tablet device which is physically proximate to the sensor system 26. The EEG representations 40 may be spectral or wavelet graphs (see FIG. 3A) superimposed onto preceding graphs from a time-shifted segment, power ratios (see FIGS. 3B-F), attractors (see FIG. 4A) generated from time-delayed embeddings created from the preprocessed EEG signals, EEG representations generated from temporal changes in relative entropy (see FIG. 4B), and/or graphs (see FIGS. 6B, 6C) illustrating a degree of synchrony or functional connectivity between two or more EEG electrodes 28. The delirium classification 44 may be a binary classification indicating whether or not delirium was likely to be present in the patient under observation based on the EEG signals 30.

The computing device 12 also comprises an output device 24 and other suitable computer components configured to implement the techniques and processes described herein. In one example, the computing device 12 may take the form of a desktop computing device, a laptop computing device, or another suitable type of computing device. In this example, the output device 24 may take the form of a display monitor, a large format display, a projector, a display integrated in a mobile device, a sound or alarm indicator, etc. The input/output module 18 may include one or more input devices, such as, for example, a keyboard, a mouse, one or more camera devices, a microphone, etc. A bus 20 may operatively couple the processing circuitry 14, the volatile memory 16, and the input/output module 18 to the non-volatile memory 22.

The non-volatile memory 22 retains instructions stored data even in the absence of externally applied power, such as FLASH memory, a hard disk, read only memory (ROM), electrically erasable programmable memory (EEPROM), etc. The instructions include one or more programs, including the application 32, the signal amplifier 31, the signal preprocessor 34, the EEG representation generator 38, the delirium classifier 42, the notification generator 46, and data used by such programs sufficient to perform the operations described herein.

The processing circuitry 14 is a microprocessor that includes one or more of a central processing unit (CPU), a graphical processing unit (GPU), specialized artificial intelligence (AI) processors, an application specific integrated circuit (ASIC), a system on chip (SOC), a field-programmable gate array (FPGA), a logic circuit, or other suitable type of microprocessor configured to perform the functions recited herein. The system 10 further includes volatile memory 16 such as random access memory (RAM), static random access memory (SRAM), dynamic random access memory (DRAM), etc., which temporarily stores data only for so long as power is applied during execution of programs.

Although the application 32 is depicted as being hosted on one computing device 12, it will be appreciated that, in a remote monitoring configuration, the application 32 can alternatively be hosted across a plurality of computing devices to which the computing device 12 is communicatively coupled via a network, which can take the form of a local area network (LAN), wide area network (WAN), wired network, wireless network, personal area network, or a combination thereof, and can include the Internet. Accordingly, the remote monitoring of EEG signals 30 may facilitate the evaluation of delirium in diverse medical settings.

The sensor system 26 of the computing device 12 comprises one or more EEG electrodes 28 or leads configured to obtain electrical EEG signals 30 corresponding to brain electrical activity from a human brain of a user. These EEG signals 30 may correspond to electrical activity from a plurality of neurons or underlying neural networks. The sensor system 26 may be configured to be hard-wired or wirelessly coupled to the computing device 12, depending on different usage scenarios and preferences.

The EEG electrodes 28 may be configured for external attachment to the scalp of a user as extra-cranial sensors, which do not require head shaving for application. In various embodiments, the EEG electrodes 28 may comprise hydrogel-based, dry conductive, metallic, or composite materials configured for mechanical contact with the scalp, such as those incorporated into wearable structures including, but not limited to, patches or headbands. In some embodiments, the electrodes 28 may include or consist of flexible or conformal bioelectronic materials, including, for example, ultrathin films, electronic tattoos, printed conductive polymers, stretchable electronics, sintered conductive materials, or other skin-conformal technologies. The electrodes 28 may further be positioned in or around the ear, including within devices such as earbuds or hearing aids, or as discrete elements conforming to the auricular region. The EEG electrodes 28 are preferably configured to maintain electrical contact with the skin while ensuring user comfort and positional stability during various postures, including standing, supine, lateral, or prone orientations, for continuous or intermittent use over durations ranging from several minutes to multiple weeks.

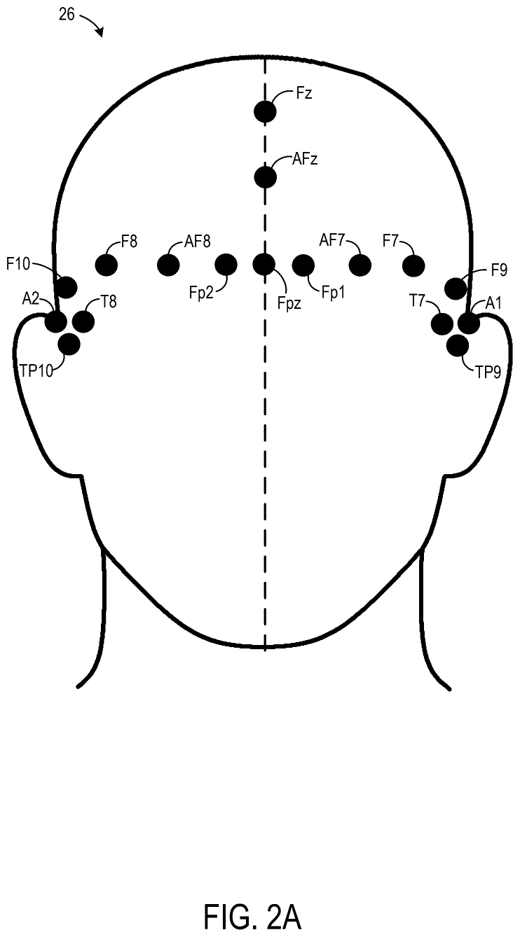

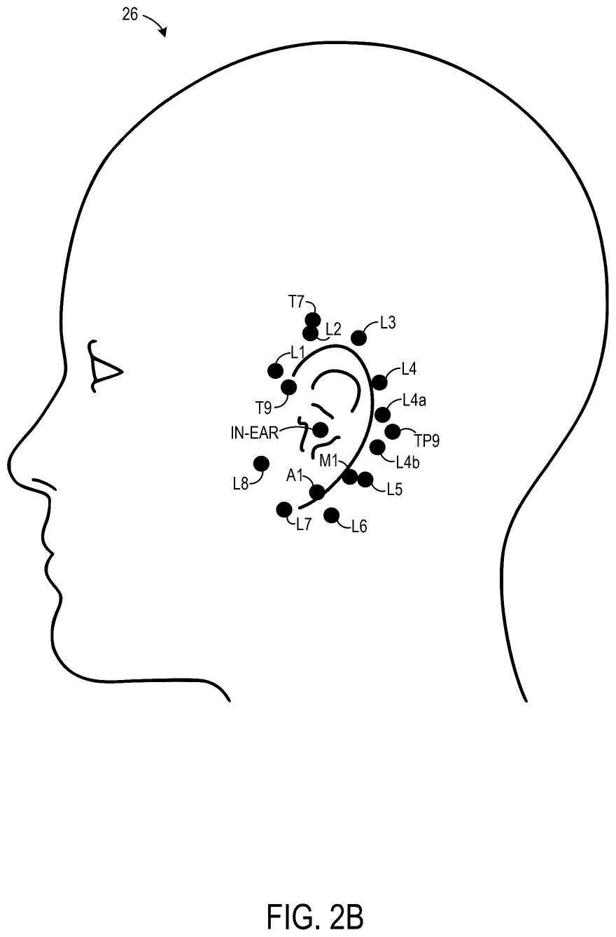

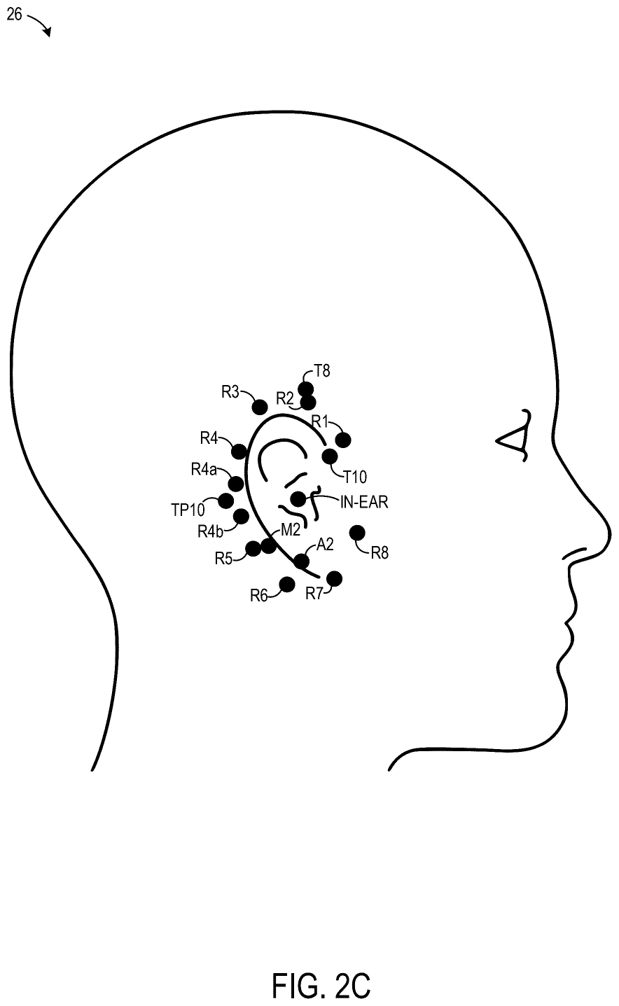

FIG. 2A shows a frontal view illustration of some of the exemplary scalp placements of the one or more EEG electrodes 28. In one example implementation, the sensor system 26 comprises only a single EEG electrode placed at the F7 position on the left frontal region of the scalp with the AFz or Fpz or A1/M1 position as a reference. Accordingly, the EEG electrode 28 may capture relevant brainwave patterns associated with delirium. Alternatively or additionally, the EEG electrode 28 may be placed in a forehead region at the Fp1 position, Fp2 position, AF7 position, AF8 position, or the F8 position on the right frontal region. Alternatively or additionally, the EEG electrode 28 may also be placed at other positions in the forehead region which lie on a margin of the temple and upper forehead: the F3 position, Fz position, or the F4 position.

As shown in the left side view illustration (FIG. 2B) and the right side view illustration (FIG. 2C) of the scalp, the EEG electrode 28 may also be placed in a periauricular region at the A1 position, A2 position, F9 position, or F10 position on the scalp. The EEG electrode 28 may also be placed at other positions in the periauricular region which lie in the mastoid region (M1 or M2 positions), in the anterior region (FT9 or FT10 positions), in the mid-temporal region (T9, T10, TP9, or TP10 positions), in the temporal edge region (T7 or T8 position), or in the posterior/postauricular region (P9, P10, PO9, PO10, O9, or O10 positions).

The EEG electrode 28 may also be placed inside a concha or ear canal as an in-ear electrode in an earpiece or earbud (ExA/B/E/G/I/K, or R1-R10/L1-L10 positions). For example, the EEG electrode 28 may be configured as a concha contact configured to contact a superior concha or an inferior concha. Alternatively, the EEG electrode 28 may be configured as an ear-canal tip contact. For example, the ear-canal tip contact may be configured as pads contacting an inside of the ear canal.

Additionally, an EEG electrode at the Fpz position, at the AFz position or A1/M1 or A2/M2 may be used as a reference point or baseline for measuring the electrical activity detect at the single main EEG electrode placed at positions in the forehead region or periauricular region on the scalp or in an in-ear region. A reference electrode serves as a comparison point for the electrical potentials recorded at the single main EEG electrode, so that a difference in electrical potential between the single main EEG electrode and the reference electrode can be measured. Accordingly, a stable baseline is provided from which to measure the electrical brainwave activity at the single main electrode.

The processing circuitry 14 executes an application 32 which receives a plurality of EEG signals 30 from the one or more EEG electrodes 28. The plurality of EEG signals 30 may be received in a spot-check mode or a continuous monitoring mode, thereby ensuring uninterrupted EEG data collection. The duration of the continuous monitoring mode is not particularly limited, and may be as short as five minutes or as long as 72 hours or more.

A signal amplifier 31 may receive the plurality of EEG signals 30 and generate amplified EEG signals 33 by increasing the amplitude of the EEG signals 30 to a level where they can be further processed and analyzed. The signal preprocessor 34 may preprocess the amplified EEG signals 33 by applying a signal filter 34a to the plurality of amplified EEG signals 33 received from the one or more EEG electrodes 28. For example, the signal filter 34a may be a bandpass filter configured to remove noise outside the frequency band of interest, remove impedance pulses, normalize the EEG signals, and remove low and high frequency artifacts.

The signal preprocessor 34 may further perform preprocessing by executing a signal segmenter 34b to segment the plurality of amplified EEG signals 33 into preprocessed EEG signals 36 which are a plurality of discrete temporal data segments, or time windows. Each segment represents a predefined time interval of EEG activity, which is preferably between 5 and 30 seconds, and more preferably 20 seconds. These intervals are preferably configured to be short enough to allow for rapid classification of likely delirium states while being long enough to ensure the reliability of the EEG data. The segmentation may be time-locked to an event, or the segmentation may be performed independently of any events. For example, segmentation may be time-locked to rapid changes in vital signs, including sudden increases in respiratory rate or heart rate, or sudden decreases in oxygen saturation.

The EEG representation generator 38 processes the temporal data segment of each of the preprocessed EEG signals 36 to extract features to generate EEG representations 40. Turning to the prophetic examples of FIG. 3, in one embodiment, the EEG representation generator 38 generates graphs 40 which represent the power spectra of the EEG signals 30 for each frequency. To generate each graph 40, a current graph may be superimposed onto a preceding graph from a time-shifted EEG segment, where the time shift is adjustable (between 5 and 30 seconds, for example). This process results in an overlay of EEG activity, highlighting changes in brainwave patterns at the particular position of the EEG electrode over the specified time delay. The EEG graph 40 may cover EEG signals 30 from the 0.5 Hz up to 100 Hz frequency range. For example, the EEG graph 40 may include EEG signals within the 0.1 Hz to 12 Hz frequency range, 4 Hz to 8 Hz frequency range, 8 Hz to 12 Hz frequency range, 12 Hz to 20 Hz frequency range, or the 20 Hz or greater frequency range. The restriction of the EEG graph 40 to a predetermined frequency range may ensure that only the most relevant data for the classification of the likely onset of delirium is analyzed.

As shown FIG. 3A, the EEG representations 40 may capture specific changes 50 in EEG frequencies that are indicative of delirium. Notably, there are increases in lower frequency waves and decreases in higher frequency waves. To predict depressed states of consciousness indicating delirium, the delirium classifier 42 may be trained to detect power spectra of the preprocessed EEG signals 36 which are characterized by increased power in the slower frequency bands below 13 Hz. Additionally or alternatively, the delirium classifier may be trained to detect an increase in the alpha range (8 to 12 Hz) with peaks in the delta range (0.1 to 4 Hz), and/or the delirium classifier may be trained to detect an increase in the ratio of the amplitude of the alpha range and delta range relative to other ranges. The delta waves are generally associated with deep sleep or severe brain pathology, while increased alpha activity can indicate a disconnection from external stimuli, often seen in delirium.

On the other hand, in conscious states of non-delirium, the power spectra of the EEG signals 30 are generally characterized by increased power in the frequency bands above 12 Hz. This may indicate normal brain activity and alertness, where faster brainwave frequencies like beta (13 to 20 Hz) and gamma (20 Hz and above) are more prominent.

In the non-limiting prophetic examples of FIG. 3A, EEG representations 40 may capture specific changes 50 in EEG frequencies that are in the delta range (0.1 to 4 Hz). In FIG. 3A, in the non-delirium state, the relative power in the delta range (0.1 to 4 Hz) of the EEG frequencies is lower than in the delirium state, in which the specific change 50 in the EEG frequencies is the increased relative delta power in the delta range frequencies of 0.1 to 4 Hz. Therefore, the delirium classifier 42 may be trained to classify EEG representations 40, in which the relative delta power is increased, as a delirium state.

In FIG. 3A, in the non-delirium state, the relative power in the theta range (4 to 8 Hz) of the EEG frequencies is also lower than in the delirium state, in which the specific change 51 in the EEG frequencies is the increased relative theta power in the theta range frequencies of 4 to 8 Hz. Therefore, alternatively, the delirium classifier 42 may be trained to classify EEG representations 40, in which the relative theta power is increased, as a delirium state.

As shown in the non-limiting prophetic examples of FIGS. 3B-F, the EEG representations 40 may capture specific changes in the ratios of the summed absolute powers of all frequencies of the EEG signals in specific EEG frequency bands. These specific changes in the ratios may be used to generate a classification 44 of delirium. Notably, the EEG representations 40 may capture the ratio of the summed absolute power of all frequencies in the alpha range (8 to 12 Hz) to the summed absolute power of all frequencies in the delta range (0 to 4 Hz) (see FIG. 3B), the ratio of the summed absolute power of all frequencies in the alpha range (8 to 12 Hz) to the summed absolute power of all frequencies in the theta range (4 to 8 Hz) (see FIG. 3C), the ratio of the summed absolute power of all frequencies in the beta range (13 to 20 Hz) to the summed absolute power of all frequencies in the delta range (0 to 4 Hz) (see FIG. 3D), the ratio of the summed absolute power of all frequencies in the beta range (13 to 20 Hz) to the summed absolute power of all frequencies in the theta range (4 to 8 Hz) (see FIG. 3E), and/or the ratio of the summed absolute power of all frequencies in the ≤12 Hz frequency range to the summed absolute power of all frequencies in the 13 Hz or greater frequency range (see FIG. 3F).

Responsive to determining a decrease in the ratio of the summed absolute power of all frequencies in the alpha range to the summed absolute power of all frequencies in the delta range, a decrease in the ratio of the summed absolute power of all frequencies in the alpha range to the summed absolute power of all frequencies in the theta range, a decrease in the ratio of the summed absolute power of all frequencies in the beta range to the summed absolute power of all frequencies in the delta range, a decrease in ratio of the summed absolute power of all frequencies in the beta range to the summed absolute power of all frequencies in the theta range, and/or an increase in the ratio of the summed absolute power of all frequencies in the 0 to 13 Hz or ≤12 Hz frequency range to the summed absolute power of all frequencies in the 13 Hz to 50 Hz frequency range, the delirium classifier 42 generates and outputs a classification 44 indicating the likely onset of delirium. The 0 to 13 Hz frequency range may be referred to as the delta plus theta plus alpha range, and the 13 Hz to 50 Hz frequency range may be referred to as the beta plus low gamma range. The generation and output of the classification 44 may be triggered by the ratio decreasing below a predetermined ratio threshold or by the ratio increasing above a predetermined ratio threshold, as illustrated by the dotted line in each of FIGS. 3B-F. Alternatively, the generation and output of the classification 44 may be triggered by the rate of increase of the ratio or the rate of decrease of the ratio exceeding a predetermined ratio change threshold.

Returning to FIG. 1A, the generated EEG representations 40 are inputted into the delirium classifier 42, which extracts a plurality of features, such as amplitude, frequency, phase information, power spectral densities, band power, connectivity measures, and other relevant EEG signal characteristics, from the EEG representations 40. These extracted features may be associated with altered attention, arousal, or cognitive fluctuation patterns. Then, a machine learning algorithm 42a may be applied to the extracted features to generate a classification 44, determining whether the brainwave patterns are indicative of delirium onset. The delirium classifier 42 may be configured as a time-continuous model which classifies EEG representations 40 in real-time as EEG signals 30 are continuously collected.

To train the machine learning algorithm 42a, a training data set 52 of EEG representations from known delirious and non-delirious states is compiled and fed into the machine learning algorithm 42a. The training data set 52 includes classifier training input data 52a and associated classifier ground truth labels 52b, which may include clinical delirium diagnoses and/or proxy clinical evaluations for delirium based on the DSM-5-TR diagnostic criteria or other diagnostic gold standards.

The processing circuitry 14 is configured to pair the classifier ground truth labels 52b with the classifier training input data 52a, and perform training of the machine learning algorithm 42a using pairs of classifier ground truth labels 52b and classifier training input data 52a in the training data set 52. For example, the EEG graphs of FIGS. 3A-B indicating delirium onset would be tagged with classifier ground truth labels 52b, ‘delirium state’, and incorporated into the training data set 52 as classifier training input data 52a. Accordingly, the machine learning algorithm 42a is trained to differentiate between delirious states and non-delirious states in the EEG representations 40.

The machine learning algorithm 42a may comprise a time-continuous machine learning model, such as a time-continuous recurrent neural network. One example of a time-continuous recurrent neural network is a liquid time-constant network (LTCN). A validation test set may be developed from the training data set 52 and then used to tune hyperparameters on the trained machine learning algorithm 42a.

When the delirium classifier 42 outputs a classification 44 that delirium is likely present in the patient based on the EEG signals 30, the notification generator 46 generates one or more notifications 48. In the example of FIG. 1A, a notification 48 indicating that delirium is likely present is outputted on the output device 24. In some embodiments, the one or more notifications 48 may be outputted to a healthcare practitioner to assess whether a subject has experienced an onset of delirium, thereby providing clinically useful diagnostic information to the healthcare practitioner. The one or more notifications 48 may be generated in the form of visual, audio, and/or textual alerts.

Additionally or alternatively, the notification 48 may be sent to an intervention device 72 configured to perform a neuro-intervention 76 to treat a delirium episode. The intervention device 72 executes logic 74 to perform the neuro-intervention 76 responsive to receiving the notification 48 that delirium has been detected. The neuro-intervention 76 may include localized or systemic delivery of therapeutic substances, ultrasonic treatment, visual stimulation, auditory stimulation, magnetic stimulation, and/or electrical stimulation. For example, transcranial magnetic stimulation (TMS) or transcranial electrical stimulation (TES) may be applied to a user by the intervention device 72 responsive to receiving the notification 48 that delirium has been detected. The ultrasonic treatment may include focused ultrasound (fUS). The auditory stimulation may include audio-visual neuromodulation. The therapeutic substances that are delivered during a neuro-intervention 76 may include oral or intravenous anti-inflammatory agents and/or neurotransmitter modulators, for example.

Turning to FIG. 1B, a second exemplary real-time delirium classification system 100 is illustrated, which generates a delirium classification 144 not only based on EEG signals 130, but also based on electronic health records 160 and/or supplemental health data 180, and the delirium classification 144 includes a confidence score 144a. In FIGS. 1A and 1B, similar parts share similar numbers, and description thereof is omitted except where different for the sake of brevity.

The delirium detection computing device 112 may be configured to be integrated with an electronic medical record (EMR) system 158, thereby enabling dynamic, context-aware delirium detection based on multimodal clinical data. Examples of EMR systems include Epic®, Cerner®, and other HL7/FHIR-compliant systems.

The delirium detection computing device 12 may be configured to interact with the EMR system 158 over a secure hospital network. By interfacing with the EMR system 158, the device 112 may periodically query, retrieve, and aggregate patient-specific electronic health records 160 in real time, including structured and unstructured entries corresponding to clinical observations, diagnostics, and patient histories, as described in further detail with respect to FIG. 1C below.

The delirium classifier 142 includes a weighted score generator 162 which is configured to receive electronic health records 160 from the EMR system 158 and/or supplemental health data 180 from non-EMR data sources 178, and generates a weighted score 168 based on the health data 160, 180 of the user. A weighted score 168 may be generated by assigning a risk weight for each variable identified in the health data 160, 180, and synthesizing the risk weights into a composite risk factor score.

The delirium classifier 142 also includes a machine learning algorithm 164 which is configured to generate a binary delirium classification 144 and a probability score 166 based on the EEG representations 140. When the machine learning algorithm 164 is configured as a recurrent neural network, the probability score 166 may be generated as a probability value by the activation function applied to the final layer of the network. The probability score 166 is in the range of 0 to 1, which indicates a degree of certainty of the machine learning algorithm 164 about the classification 144. The weighted score 168, the classification 144, and the probability score 166 are received as input by the classification generator 170 of the delirium classifier 142 to generate a classification 144 with a confidence score 144a which takes into account the probability score 166 from the machine learning algorithm 164 and the weighted score 168 from the weighted score generator 162.

In some embodiments, the machine learning algorithm 164, configured as a recurrent neural network, may additionally generate a severity score 167. The severity score 167 may be a score ranging from 0 to 39 in accordance with the Delirium Rating Scale-Revised-98 (DRS-R-98), a score ranging from 0 to 30 in accordance with the Memorial Delirium Assessment Scale (MDAS), a score ranging from 0 to 7 or from 0 to 19 in accordance with the Confusion Assessment Method-Severity (CAM-S), a score ranging from 0 to 8 in accordance with the Intensive Care Delirium Screening Checklist (ICDSC), or a score ranging from 1 to 3 (1 for mild, 2 for moderate, and 3 for severe) in accordance with DSM-5-TR. The machine learning algorithm 164 may be configured with a second activation function to output the severity score 167 indicating the severity of the delirium classification 144. Accordingly, the machine learning algorithm 164 may be trained delirium classifier training data 152, in which the classifier training input data 152a and associated classifier ground truth labels 152b include clinical delirium diagnoses and/or proxy clinical evaluations with severity scores for delirium based on the diagnostic criteria described above. The weighted score 168, the classification 144, the probability score 166, and the severity score 167 may be received as input by the classification generator 170 of the delirium classifier 142 to generate a classification 144. The classification 144 may include a severity score 169 which takes into account the severity score 167 generated by the machine learning algorithm 164 and the weighted score 168 generated by the weighted score generator 162. The classification 144 may also include a confidence score 144a which takes into account the probability score 166 generated by the machine learning algorithm 164 and the weighted score 168 generated by the weighted score generator 162.

The notification generator 146 generates one or more notifications 148, which are outputted on the output device 124. In this example, not only does the notification 148 indicate that delirium has been detected, but also indicates the confidence score 144a, which is 70% in this example. In some embodiments, the notification may also include the severity score 169 indicating the severity of the delirium classification 144, which is a severity score 169 of twenty in this example. The notification 148 is also sent to an intervention device 72, which executes logic 74 to determine whether the confidence score 144a exceeds a predetermined confidence score threshold. Responsive to determining that the confidence score 144a exceeds the predetermined confidence score threshold, the intervention device 72 performs a neuro-intervention 76 to treat a delirium episode. The predetermined confidence score threshold is not particularly limited, and may be configured as 60%, 75%, or 90% for example.

FIG. 1C illustrates a non-limiting example of the types of data that may be included in the electronic health record 160 or the supplemental health data 180 illustrated in the second system 100 of FIG. 1B. The health data 158, 180 may comprise biometric monitoring data 160a, which may include heart rate (HR), non-invasive or invasive blood pressure (BP), central venous pressure (CVP), respiratory rate (RR), oxygen saturation (SpO₂), intracranial pressure (ICP), accelerometry (3-axis), pupillary diameter (via pupillometry), eye tracking and movement data, facial skin color (via facial recognition imaging or RGB/thermal sensors), expired CO₂ concentration (EtCO₂), and/or audio-derived vocalizations. The biometric monitoring data 160a may include biometric statistical data, which may encompass the mean, standard deviation, velocity, acceleration (rate of change of the velocity), and area under the curve of the HR, BP, RR, SpO₂, accelerometry, pupillary diameter, eye tracking and movement data, facial skin color, and/or EtCO₂. For example, biometric statistics may include heart rate variability, the acceleration of blood pressure change on the descent of the arteria line waveform, or stroke volume.

The health data 158, 180 may also comprise patient history data 160b including patient age, frailty score, diagnosis or suspicion of dementia (including Alzheimer's disease), mobility status (with or without assistance), activities of daily living (ADL) independence, history of cerebrovascular accident (CVA/stroke), history of acute or chronic kidney disease (AKI, CKD), diabetes mellitus (DM), coronary artery disease (CAD), documented substance abuse, prior delirium episodes, history of traumatic brain injury (TBI), history of anoxic brain injury, and/or discharge disposition prior to admission (e.g., skilled nursing facility, assisted-living, unhoused).

The health data 158, 180 may also comprise medication profile data 160c including the use of psychotropic agents, sedative-hypnotic agents, and/or analgesic/pain medication usage. The health data 158, 180 may also comprise physical state data 160d including presence of sepsis or systemic infection, history of falls with loss of consciousness, acute diagnoses such as TBI, anoxic brain injury, acute kidney injury (AKI), stroke (ischemic or hemorrhagic), and/or cardiac event.

The health data 158, 180 may also comprise the environmental context 160e of the patient including post-operative status, including type and severity of surgeries, in-hospital location and level of care (whether the patient is in an intensive care unit (ICU), intubation status, medical or surgical hospital ward, emergency department (ED)), and/or time proximity between procedure and current delirium symptoms.

The health data 158, 180 may also comprise laboratory data 160f including genomic and epigenomic sequencing data, brain imaging scans (e.g., MRI, CT, PET) with findings indexed via natural language processing, inflammatory biomarkers such as IL-6, TNF-alpha, and CRP, CSF analysis results, metabolic panels, glucose monitoring data, and/or drug screens. The laboratory data 160f may include laboratory statistical data, which may encompass the mean, standard deviation, velocity, acceleration (rate of change of the velocity), and area under the curve of the glucose monitoring data. For example, the laboratory statistical data may include an area under the curve for continuous glucose monitoring data, which may indicate how elevated serum glucose levels have been and for how long.

The health data 158, 180 may also comprise vigilance and cognitive state data 160g including sleep-wake cycles and circadian rhythm markers, sleep stage classification (REM, NREM I-III), awake attentional and engagement state (e.g., sustained attention, stupor, coma), and/or Richmond Agitation-Sedation Scale (RASS) score.

Turning to FIG. 1D, non-limiting examples of the different modalities of the notifications 48 outputted by the output device 24 of the first system 10 or the notifications 148 outputted by the output device 124 of the second system 100 are illustrated. In these examples, the visual notification 148 is an indication that delirium has been detected and the confidence score 44a, 144a associated with the delirium classification 144. In FIG. 1D, colors indicate the severity score 167, with colors that are greener indicate lower severity scores, and colors that are redder indicate higher severity scores. The output device 124 also outputs an audio notification 149 indicating the severity score 167, with higher musical pitches or sound frequencies indicating lower severity scores, and lower musical pitches or sound frequencies indicating higher severity scores. Alternatively, the audio notification 149 may similarly indicate the confidence score 44a, 144a.

It will be understood that the visual notifications 148 and audio notifications 149 outputted by the output devices 24, 124 are non-limiting, and that numerous alternative or additional notification modalities may be employed. For example, the visual notification 148 may include progress bars, renderings of numeric or percentage values, pattern changes, or overlay textual descriptors to indicate the confidence score 44a, 144a or severity score 167. The audio notification 149 may comprise a sequence of tones where rhythm or interval spacing encodes the confidence score 44a, 144a or severity score 167.

Turning to FIG. 4A, in other embodiments, the EEG representations 40 generated by the EEG representation generator 38 may be attractors 40 generated from time-delayed embeddings observed in three-dimensional state space which are created from the preprocessed EEG signals 36. Attractors 40 are patterns that the state of a dynamic system tends to evolve toward. In the context of EEG analysis, these attractors 40 of time-delayed embeddings represent EEG signals 30 over time, providing a detailed analysis of brain activity patterns.

The delirium classifier 42 may determine whether the attractors 40 are indicative of a delirium state by employing a geometric phase-space analysis, fitting the three-dimensional attractor with an ellipsoidal solid of revolution, and then calculating the ratio between the major and minor axes of this ellipsoidal solid. When a significant and consistent change in the ellipse radius ratio is detected, in which the initially spherical attractors 40 become flattened and more ellipsoidal, then a depressed state of consciousness indicating delirium is predicted. In the non-limiting prophetic example of FIG. 4A, the substantially spherical attractor 40 in the non-delirium state becomes a flattened, ellipsoidal attractor 40 in the delirium state. A predetermined threshold may be set for changes in the ellipse radius ratio. For example, when the delirium classifier 42 detects changes in the ellipse radius ratio of the attractor 40 above a predetermined threshold, then the delirium classifier 42 may output a delirium state classification 44. The predetermined threshold may be modified based on biometrics data and/or a medical history of the user.

Alternatively, the delirium classifier 42 may execute the machine learning algorithm 42a to classify the changes in the ellipse radius ratio as indicative of a delirium or not. The EEG representation generator 38 may generate three-dimensional attractors 40 which represent time-delayed embeddings observed in three-dimensional state space. A current attractor may be superimposed onto a preceding attractor from a time-delayed segment, where the time delay is adjustable (between 10 and 30 seconds, for example). The superimposed attractors 40 may be inputted into the delirium classifier 42, which extracts a plurality of features from the superimposed attractors 40. The machine learning algorithm 42a may be applied to the extracted features to generate a classification 44, determining whether the brainwave patterns are indicative of delirium onset. To train the machine learning algorithm 42a, a training data set 52 of superimposed attractors 40 from known delirious and non-delirious states may be compiled and fed into the machine learning algorithm 42a. Accordingly, the machine learning algorithm 42a may be trained to differentiate between delirious states and non-delirious states in the superimposed attractors 40, which represent changes in the ellipse radius ratios of the attractors 40.

Referring to FIG. 4B, a non-limiting prophetic example is shown of another entropy-based measure, which quantifies the complexity of an EEG signal 30 by capturing the nonlinear, complex, and irregular dynamics of EEG signals 30 over time. As patients without delirium tend to show more entropy or complexity in the EEG signals 30, and patients without delirium show less entropy or complexity in the EEG signals 30, the entropy of the EEG signals 30 may be monitored as the EEG representations 40. Examples of the EEG representations 40 as entropy-based measures include, but are not limited to, time-delayed embeddings, which create dynamical three-dimensional state attractors, sample entropy (SampEn), fuzzy entropy (FuzzyEn), multi-scale entropy (MSE), multi-scale fuzzy entropy (MFE), refined composite multiscale fuzzy entropy (RCMFE). SampEn, MSE, MFE, and RCMFE may be calculated on EEG time-series data by evaluating the entropy across multiple temporal scales.

To generate the EEG representations 40 as entropy-based measures, the received EEG signals 30 may be segmented into discrete temporal data segments to generate preprocessed EEG signals 36. Then the EEG representations 40 may be generated from temporal changes in relative entropy calculated from the preprocessed EEG signals 36. The delirium classifier 42 may determine whether EEG representations 40 are indicative of a delirium state by employing relative entropy to the EEG time-series data along multiple temporal scale as shown in FIG. 4B, which depicts the EEG representations 40 which are generated from the temporal changes in relative entropy calculated from the preprocessed EEG signals 36. Responsive to determining a decrease in the relative entropy over time, the delirium classifier 42 generates and outputs a classification 44 indicating the likely onset of delirium. The generation and output of the classification 44 may be triggered by the entropy decreasing below a predetermined entropy threshold.

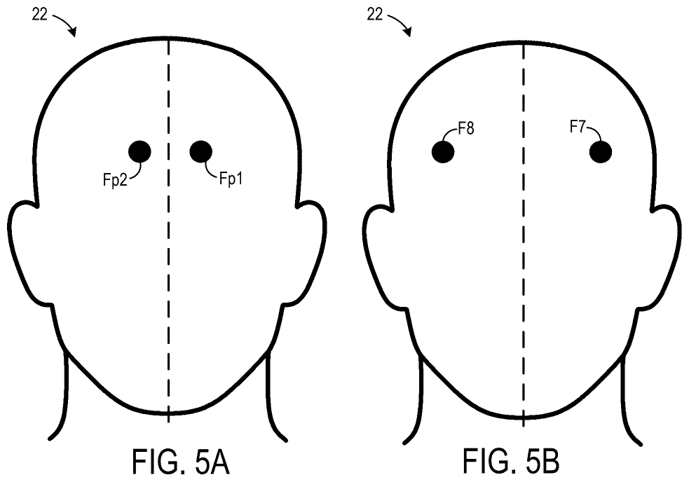

Referring to FIGS. 5A-C, additionally or alternatively to the single EEG electrode configuration described in FIGS. 2A-D, two or more EEG electrodes 28 may be used in the sensor system 26 instead of a single electrode 28 to enhance the classification of likely delirium through the monitoring of bilateral interhemispheric synchrony. The two or more EEG electrodes 28 are strategically placed on specific bilateral locations on the scalp. They can be positioned either at the Fp1 position and the Fp2 position (see FIG. 5A), which are located on the frontal poles, or at the F7 position and the F8 position (see FIG. 5B), which are situated on the lateral frontal areas. These locations are chosen for their relevance in capturing brainwave activity indicative of delirium. The bilateral placement of these electrodes 28 assists in the monitoring the synchrony and connected strength between the two hemispheres of the brain, particularly in the low-frequency bands (0.1 to 12 Hz), which includes a range from delta to alpha waves. This frequency range is significant as it is often implicated in states of reduced consciousness and delirium.

The EEG signals 30 from both electrodes 28 may be band-pass filtered by the signal filter 34a between 0.1 and 4 Hz, 4 to 8 Hz, 8 to 12 Hz, 12 to 20 Hz, and/or 20 Hz or greater, thereby isolating the relevant frequency ranges and enhancing the accuracy of synchrony or connected strength detection. In each 0.1 second time bin, local minima in the EEG signal 30 may be identified and counted. Local minima are points where the signal reaches a low point, which help clarify the wave pattern and synchrony of the EEG signals 30.

Referring to FIG. 6A, a schematic representation is shown of a pair of EEG electrode positions (Fp1 and Fp2 positions) which may be used to measure a degree of synchrony or connectivity for determining a non-delirium state or a delirium state. Referring to FIG. 6B, a non-limiting prophetic example is depicted of an EEG representation 40 which is generated as a graph indicating a degree of synchrony over time between two or more EEG electrodes 28 (between Fp1 and Fp2 in the example of FIG. 6B). The degree of synchrony between the two or more leads (electrodes) may be calculated and quantified by the EEG representation generator 38, with values ranging from 0 to 1, to generate the EEG representation 40. The horizontal axis of the graph represents time, measured in seconds, while the vertical axis quantifies the degree of synchrony or connected strength. A value closer to 1 indicates a higher degree of synchrony or connection between the hemispheres, while a value closer to 0 indicates less synchrony or connection.

A sudden decrease 54 in this bilateral interhemispheric synchrony or strength, particularly in a previously conscious user, may be indicative of a transition to a depressed state of consciousness suggesting the onset of delirium. When the delirium classifier 42 determines a sudden decrease 54 in this bilateral interhemispheric synchrony or strength, the delirium classifier 42 may generate and output a classification 44 indicating the likely onset of delirium. The sudden decrease 54 may be determined by calculating whether the negative change 54 in the synchrony or strength per unit of time is greater than or equal to a predetermined synchrony or strength change threshold. The predetermined synchrony change threshold may be modified based on biometrics data and/or a medical history of the user. In this non-limiting prophetic example, a degree of synchrony, which was value A between the two or more EEG electrodes 28, drops to value B after the sudden decrease 54 in bilateral interhemispheric synchrony.

Referring to FIG. 6C, the degree of functional connectivity between the two or more leads (electrodes) may be calculated and quantified by the EEG representation generator 38 to generate an EEG representation 40 which is a graph indicating a degree of functional connectivity between the two or more EEG electrodes 28. The horizontal axis of the graph represents time, while the vertical axis quantifies the degree of functional connectivity. The dotted line indicates the functional connectivity for the non-delirium state, while the solid black line indicates the functional connectivity for the delirium state.

A weighted phase lag index (wPLI), or the cross-spectrum of every pair of channels, may be used to calculate the functional connectivity strength. To calculate the wPLI between pairs of EEG electrodes, the EEG signals 33 are first filtered by the signal preprocessor 34 into frequency bands (delta, theta, alpha, beta, and gamma, for example) and segmented by the signal segmenter 34b into short, clean epochs. Then, each epoch is transformed into the frequency domain using a Fourier transform, and the cross-spectrum between each electrode pair is computed. The wPLI may be calculated from the imaginary part of the cross-spectrum, capturing how consistently one signal leads or lags the other across time, while excluding zero-lag (volume-conducted signals). The result is a value between 0 and 1, where higher values indicate stronger, true functional connectivity.

The wPLI may be based on the Hilbert transformed instantaneous phase differences (Δφ), capturing the asymmetry in phase leading and lagging between two signals. The weighted phase lag index wPLI=| [symbol] sign[sin(Δ_{φ}(t))] [symbol] |, where the sign[Δ_{φ}(t)] is 1 for all positive phase differences and −1 for all negative phase differences, which are averaged over an epoch. wPLI values range between 0 and 1; 1 indicates complete phase-locking and values reaching 0 indicate no phase synchronization or equal in leading and lagging over the epoch.

Functional connectivity may be quantified as a renormalized partial directed coherence (rPDC), which utilizes multivariate autoregression as a measure of Granger causality, in order to determine causal relationships between EEG signals 30 recorded at two or more EEG electrode positions. rPDC quantifies the frequency and strength of connectivity between an electrode pair in both directions independently. For example, for the Fp1 and F7 EEG electrode position pair, the frequency and strength of connectivity for the frontal to frontotemporal (Fp1 to F7) direction and the frontotemporal to frontal (F7 to Fp1) direction can be quantified independently of each other. Likewise, for the Fp1 and AFz EEG electrode position pair or the Fp1 and Fpz EEG electrode position pair, the frequency and strength of connectivity for the frontal to central direction and the central to frontal direction can be quantified independently of each other.

The delirium classifier 42 is executed to generate a classification indicating whether or not delirium is likely, based on the graph illustrating the degree of functional connectivity, classifying likely delirium by detecting an increase in functional connectivity in the delta range and a decrease in functional connectivity in the alpha range. When the delirium classifier 42 determines an increase in functional connectivity in the delta range, a decrease in functional connectivity in the alpha range, an increase in functional connectivity in a theta range, and a decrease in functional connectivity in a beta range, as illustrated in the non-limiting prophetic example of FIG. 6C, the delirium classifier 42 may generate a classification 44 indicating the likely onset of delirium. The notification generator 46 generates one or more notifications 48 based on the generated classification 44, and the one or more notifications 48 are outputted at an output device 24 or an intervention device 72.

Referring to the non-limiting prophetic example of FIG. 6D, illustrations are shown of the EEG representations of FIG. 1A or 1B as bar graphs indicating changes in global frontal functional connectivity between two or more EEG electrodes across a plurality of frequency ranges, including a theta range, an alpha range, and a beta range. The white bars indicate the range of wPLI values that may be observed in patients in a non-delirium state for a given frequency range, and black bars indicate the range of wPLI values that may be observed in patients in a delirium state for the given range. In this example, the normalized mean wPLI shows a widespread reduction in patients with delirium in the alpha range and the beta range, while an increase in connectivity is seen in the theta range.

This global alpha disconnectivity suggests a breakdown in coordinated brain activity, particularly affecting regions involved in attention, wakefulness, and cognitive control. The loss of alpha synchronization likely reflects impaired large-scale communication across the brain during delirium.

Beta oscillations are typically linked to active cognitive engagement, sensorimotor integration, and maintaining cognitive stability. The regional beta disconnectivity observed in delirium suggests a disruption in local cognitive control networks, which may underlie symptoms such as poor motor coordination, disorganized behavior, or fluctuating arousal levels.

On the other hand, the increase in connectivity in the theta range may reflect abnormal increases in synchronous low-frequency activity between brain areas involved in working memory, attention, and internal monitoring. In the context of delirium, the elevated wPLI values in the theta range may represent a dysregulated or compensatory mechanism that contributes to cognitive symptoms such as distractibility, disorientation, and impaired memory.

Therefore, responsive to determining a decrease in connectivity in the alpha range, a decrease in connectivity in the beta range, or an increase in connectivity in the theta range, the delirium classifier 42 generates and outputs a classification 44 indicating the likely onset of delirium. The generation and output of the classification 44 may be triggered by the connectivity decreasing below a predetermined connectivity threshold for connectivity measurements in the alpha range or the beta range, or by the connectivity increasing above a predetermined connectivity threshold for connectivity measurements in the theta range.

Referring to the non-limiting prophetic example of FIG. 6E, an illustration is shown of the EEG representations of FIG. 1A or 1B as a graph indicating changes in a degree of frontal to frontotemporal connectivity between two EEG electrodes placed at the Fp1 and F7 positions, respectively, across a plurality of frequency ranges. As shown in FIG. 6E, decreases in connectivity are demonstrated in the delta range, the theta range, and the alpha range, while increases in connectivity are demonstrated in the gamma range. Therefore, responsive to determining a decrease in frontal to frontotemporal connectivity in the delta range, the theta range, and the alpha range, and an increase in frontal to frontotemporal connectivity in the gamma range, the delirium classifier 42 generates and outputs a classification 44 indicating the likely onset of delirium. The generation and output of the classification 44 may be triggered by the connectivity, which may be measured as a wPLI value, decreasing below a predetermined connectivity threshold for connectivity measurements in the delta range, the theta range, and the alpha range, and by the connectivity increasing above a predetermined connectivity threshold for connectivity measurements in the gamma range.

Referring to the non-limiting prophetic example of FIG. 6F, an illustration is shown of the EEG representations of FIG. 1A or 1B as a graph indicating changes in a degree of frontal to central connectivity between two EEG electrodes placed at the Fp1 and Fpz positions or the Fp1 and AFz positions, respectively, across a plurality of frequency ranges. As shown in FIG. 6F, decreases in connectivity are demonstrated in the delta range, the theta range, the alpha range, the beta range, and the gamma range. Therefore, responsive to determining a decrease in frontal to central connectivity in the delta range, the theta range, the alpha range, the beta range, and the gamma range, the delirium classifier 42 generates and outputs a classification 44 indicating the likely onset of delirium. The generation and output of the classification 44 may be triggered by the connectivity, which may be measured as a wPLI value, decreasing below a predetermined connectivity threshold for connectivity measurements in the delta range, the theta range, the alpha range, the beta range, and the gamma range.

Referring to FIG. 6G, an illustration is shown of the EEG representations of FIG. 1A or 1B as a graph indicating changes in a degree of central to frontal connectivity between two EEG electrodes placed at the Fp1 and Fpz positions or the Fp1 and AFz positions, respectively, across a plurality of frequency ranges. As shown in FIG. 6G, decreases in connectivity are demonstrated in the delta range, the alpha range, the beta range, and the gamma range. Therefore, responsive to determining a decrease in central to frontal connectivity in the delta range, the alpha range, the beta range, and the gamma range, the delirium classifier 42 generates and outputs a classification 44 indicating the likely onset of delirium. The generation and output of the classification 44 may be triggered by the connectivity, which may be measured as a wPLI value, decreasing below a predetermined connectivity threshold for connectivity measurements in the delta range, the alpha range, the beta range, and the gamma range.

It will be understood that any graphical representations of the lines in FIGS. 3A, 4B, 6B, 6C, and 6E-G are provided solely for the purpose of example and illustration. These representations are not intended to limit the scope of the invention to the specific shapes depicted. In various embodiments, EEG representations may be represented by lines of different shapes.

Although various embodiments described herein refer specifically to the detection of delirium, it will be appreciated that other embodiments of the disclosed systems and methods may also be used to detect, classify, or monitor a variety of brain states or cognitive conditions, including but not limited to major or mild neurocognitive disorders, acute or subacute neurological dysfunction, neurological impairment, sedation, oversedation, confusion, encephalopathy, stroke, brain failure, or general brain health degradation.

For example, the delirium classification system 10 or 100 may be configured as a brain state detection computing system, comprising a sensor system comprising one or more electroencephalography (EEG) electrodes configured to generate a plurality of EEG signals, processing circuitry, and a non-volatile memory storing executable instructions that, in response to execution by the processing circuitry, cause the processing circuitry to receive the plurality of EEG signals, preprocess the received plurality of EEG signals to generate preprocessed EEG signals, extract features to generate EEG representations based on the preprocessed EEG signals, execute a brain state classifier to generate a classification of a predetermined brain state, based on the generated EEG representations, and generate and output one or more notifications based on the generated classification. The predetermined brain state may be at least one of a neurocognitive disorder, neurological dysfunction, a neurological impairment, sedation, oversedation, confusion, encephalopathy, stroke, brain failure, or general brain health degradation. Similar to the severity score training data discussed elsewhere herein, training data for classification of each of these predetermined brain states can be assembled using established diagnostic protocols and criteria, such as the DSM, as well as clinical evaluations, imaging, etc. A training dataset assembled in this manner includes EEG signals of brainwave activity, labeled with clinical diagnosis of the predetermined brain state, and the classifier is trained on this ground truth data.

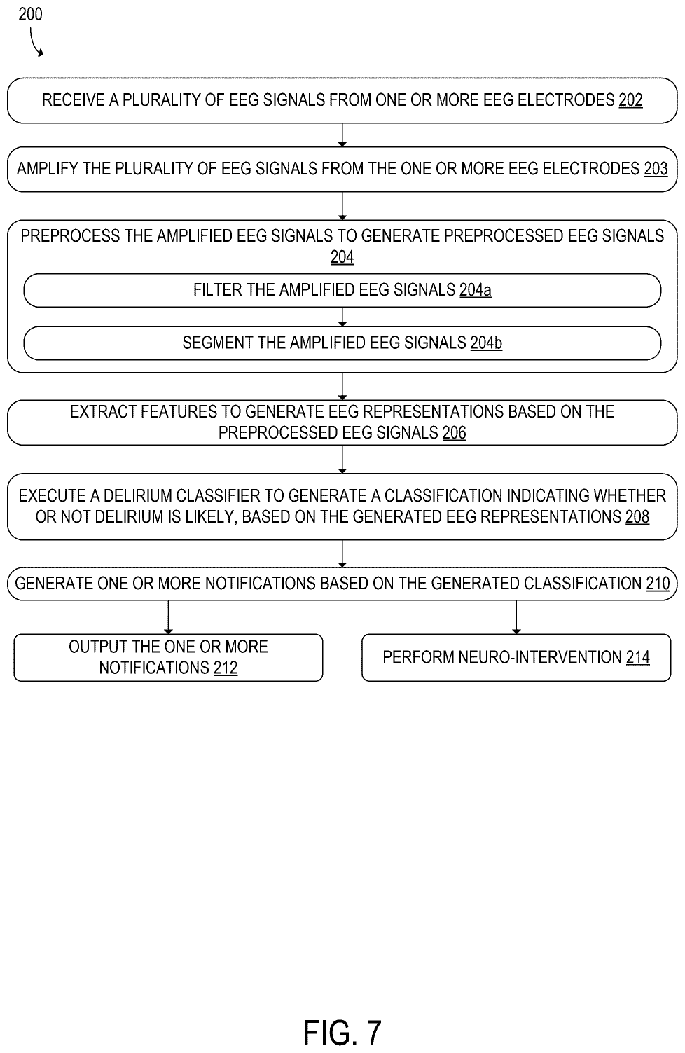

Referring to FIG. 7, a flow chart is illustrated of a first method 200 for real-time delirium classification. The first method 200 may be implemented on the delirium classification computing system 10 illustrated in FIG. 1A above, which includes the delirium classification computing device 12 and sensor system 26. Alternatively, other suitable computing hardware and software may be utilized.

At 202, the method includes receiving a plurality of EEG signals from one or more EEG electrodes. At 203, the method includes amplifying the plurality of EEG signals from the one or more EEG electrodes. At 204, the method includes preprocessing the amplified EEG signals to generate preprocessed EEG signals. Step 204 may include, at 204a, filtering the amplified EEG signals, and, at 204b, segmenting the amplified EEG signals. At 206, the method includes extracting features to generate EEG representations based on the preprocessed EEG signals. At 208, the method includes executing a delirium classifier to generate a classification indicating whether or not delirium is likely, based on the generated EEG representations. At 210, the method includes generating one or more notifications based on the generated classification. At 212, the method includes outputting the one or more notifications. The method may also include, at 214, performing a neuro-intervention based on the generated classification. The neuro-intervention may include localized or systemic delivery of therapeutic substances, ultrasonic treatment, visual stimulation, auditory stimulation, magnetic stimulation, and/or electrical stimulation.

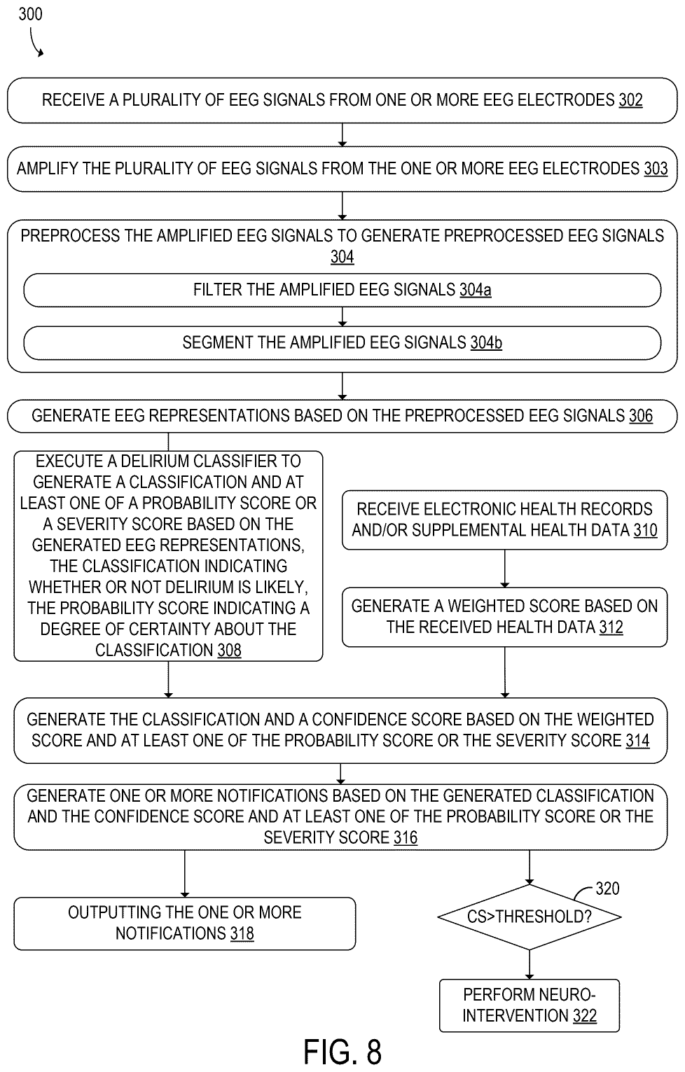

Referring to FIG. 8, a flow chart is illustrated of a second method 300 for real-time delirium classification. The second method 300 may be implemented on the delirium classification computing system 100 illustrated in FIG. 1B above, which includes the delirium classification computing device 112, sensor system 126, and a weighted score generator 162 configured to receive electronic health records 160 and/or supplemental health data 180 from non-EMR data sources 178. Alternatively, other suitable computing hardware and software may be utilized.

At 302, the method includes receiving a plurality of EEG signals from one or more EEG electrodes. At 303, the method includes amplifying the plurality of EEG signals from the one or more EEG electrodes. At 304, the method includes preprocessing the amplified EEG signals to generate preprocessed EEG signals. Step 304 may include, at 304a, filtering the amplified EEG signals, and, at 304b, segmenting the amplified EEG signals. At 306, the method includes generating EEG representations based on the preprocessed EEG signals. At 308, the method includes executing a delirium classifier to generate a classification and at least one of a probability score or a severity score based on the generated EEG representations, the classification indicating whether or not delirium is likely and the probability score indicating a degree of certainty about the classification.

At 310, the method includes receiving electronic health records from an EMR system and/or receiving supplemental health data from non-EMR sources, and at 312, the method includes generating a weighted score based on the received electronic health records and/or received supplemental health data.

At 314, the method includes generating a classification and a confidence score based on the weighted score and at least one of the probability score or the severity score. At 316, the method includes generating one or more notifications based on the generated classification and the confidence score and at least one of the probability score or the severity score. At 318, the method includes outputting the one or more notifications. The method may also include, at 320, determining whether the confidence score or the severity score is above a predetermined score threshold, and at 322, responsive to determining that the confidence score or the severity score is above the predetermined score threshold, performing a neuro-intervention, which may include localized or systemic delivery of therapeutic substances, ultrasonic treatment, visual stimulation, auditory stimulation, magnetic stimulation, and/or electrical stimulation.

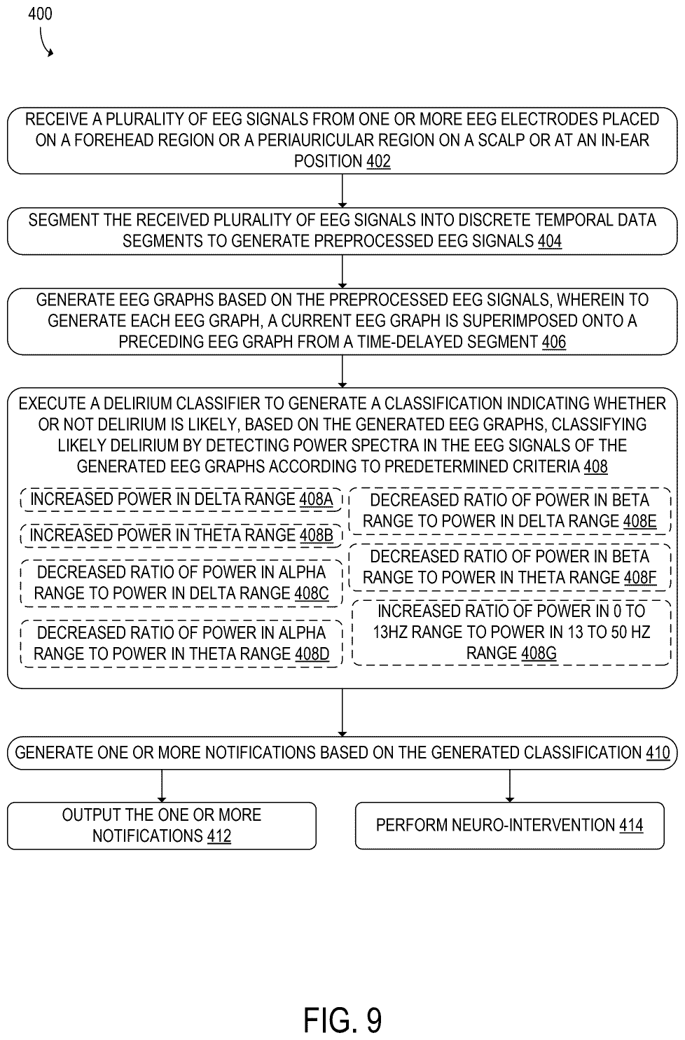

Referring to FIG. 9, a flow chart is illustrated of a third method 400 for real-time delirium classification. The third method 400 may be implemented on the delirium classification computing system 10 illustrated in FIG. 1A above, which includes the delirium classification computing device 12 and sensor system 26. Alternatively, other suitable computing hardware and software may be utilized.

At 402, the method includes receiving a plurality of EEG signals from one or more EEG electrodes placed on a position on a forehead region or a periauricular region on a scalp or at an in-ear position. At 404 the method includes segmenting the received plurality of EEG signals into discrete temporal data segments to generate preprocessed EEG signals. At 406, the method includes generating power spectrum graphs based on the preprocessed EEG signals, wherein to generate each power spectrum graph, a current power spectrum graph is superimposed onto a preceding EEG graph from a time-delayed segment. At 408, the method includes executing a delirium classifier to generate a classification indicating whether or not delirium is likely, based on the generated EEG graphs, classifying likely delirium by detecting power spectra in the EEG signals of the generated graphs according to predetermined criteria.

The predetermined criteria may include at least one of a first condition 408A of increased power in a delta range; a second condition 408B of increased power in a theta range; a third condition 408C of decreased ratio of a power in an alpha range to a power in the delta range; a fourth condition 408D of decreased ratio of power in an alpha range to power in the theta range; a fifth condition 408E of decreased ratio of power in a beta range to power in the delta range; a sixth condition 408F of decreased ratio of power in a beta range to power in the theta range; or a seventh condition of 408G increased ratio of power in the delta plus theta plus alpha (0 to 13 Hz) frequency range to power in the beta plus low gamma (13 Hz to 50 Hz) frequency range.

At 410, the method includes generating one or more notifications based on the generated classification. At 412, the method includes outputting the one or more notifications. The method may also include, at 414, performing a neuro-intervention based on the generated classification. The neuro-intervention may include localized or systemic delivery of therapeutic substances, ultrasonic treatment, visual stimulation, auditory stimulation, magnetic stimulation, and/or electrical stimulation.

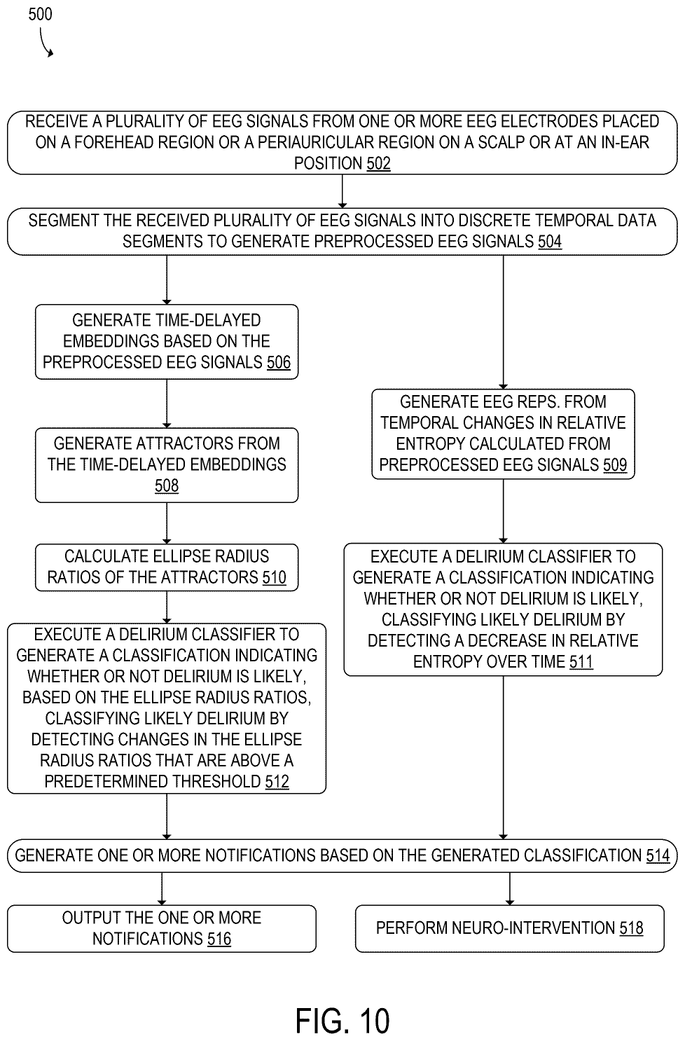

Referring to FIG. 10, a flow chart is illustrated of a fourth method 500 for real-time delirium classification. The fourth method 500 may be implemented on the delirium classification computing system 10 illustrated in FIG. 1A above, which includes the delirium classification computing device 12 and sensor system 26. Alternatively, other suitable computing hardware and software may be utilized.

At 502, the method includes receiving a plurality of EEG signals from one or more EEG electrodes placed on a position on a forehead region or a periauricular region on a scalp or at an in-ear position. At 504 the method includes segmenting the received plurality of EEG signals into discrete temporal data segments to generate preprocessed EEG signals. At 506, the method includes generating time-delayed embeddings based on the preprocessed EEG signals. At 508, the method includes generating attractors from the time-delayed embeddings. At 510, the method includes calculating ellipse radius ratios of the attractors. At 512, the method includes executing a delirium classifier to generate a classification indicating whether or not delirium is likely, based on the ellipse radius ratios, classifying likely delirium by detecting changes in the ellipse radius ratios that are above a predetermined threshold.

Additionally or alternatively, subsequent to generating discrete temporal data segments at 504, the method may proceed to 509 to generate EEG representations from temporal changes in relative entropy calculated from the preprocessed EEG signals, and 511 of executing a delirium classifier to generate a classification indicating whether or not delirium is likely by detecting a decrease in relative entropy over time.

At 514, the method includes generating one or more notifications based on the generated classification. At 516, the method includes outputting the one or more notifications. The method may also include, at 518, performing a neuro-intervention based on the generated classification. The neuro-intervention may include localized or systemic delivery of therapeutic substances, ultrasonic treatment, visual stimulation, auditory stimulation, magnetic stimulation, and/or electrical stimulation.

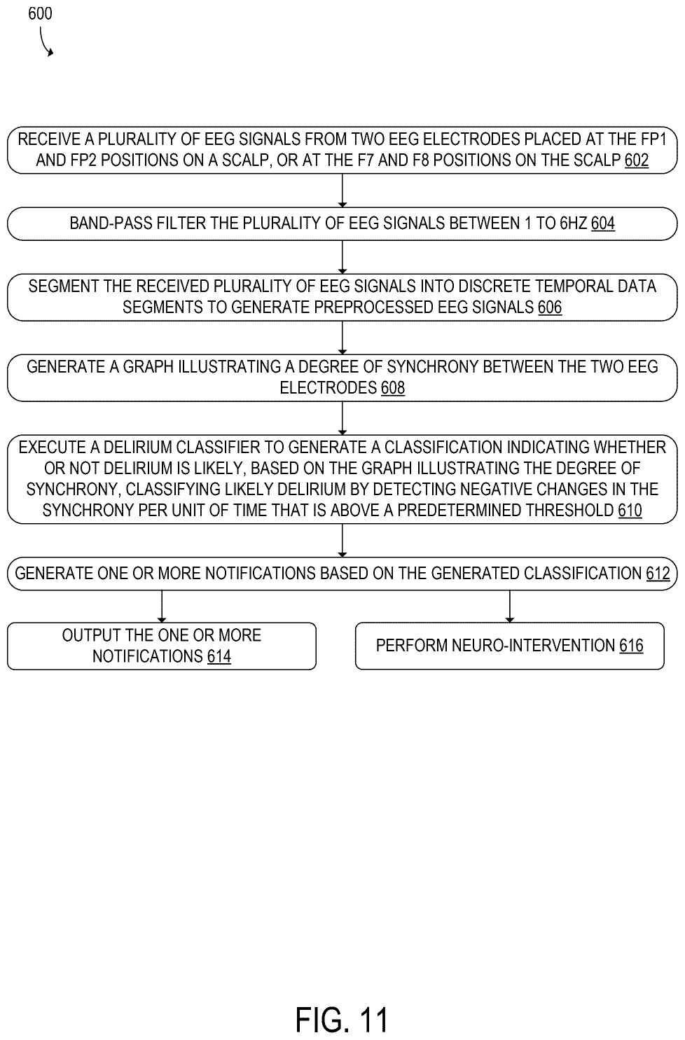

Referring to FIG. 11, a flow chart is illustrated of a fifth method 600 for real-time delirium classification. The fifth method 600 may be implemented on the delirium classification computing system 10 illustrated in FIG. 1A above, which includes the delirium classification computing device 12 and sensor system 26. Alternatively, other suitable computing hardware and software may be utilized.

At 602, the method includes receiving a plurality of EEG signals from two EEG electrodes placed at the Fp1 and Fp2 positions on a scalp, or at the F7 and F8 positions on the scalp. At 604, the method includes band-pass filtering the plurality of EEG signals between 1 to 6 Hz. At 606, the method includes segmenting the received plurality of EEG signals into discrete temporal data segments to generate preprocessed EEG signals. At 608, the method includes generating a graph illustrating a degree of synchrony or connected strength between the two EEG electrodes. At 610, the method includes executing a delirium classifier to generate a classification indicating whether or not delirium is likely, based on the graph illustrating the degree of synchrony or strength, classifying likely delirium by detecting negative changes in the synchrony or strength per unit of time that is above a predetermined threshold. At 612, the method includes generating one or more notifications based on the generated classification. At 614, the method includes outputting the one or more notifications. The method may also include, at 616, performing a neuro-intervention based on the generated classification. The neuro-intervention may include localized or systemic delivery of therapeutic substances, ultrasonic treatment, visual stimulation, auditory stimulation, magnetic stimulation, and/or electrical stimulation.

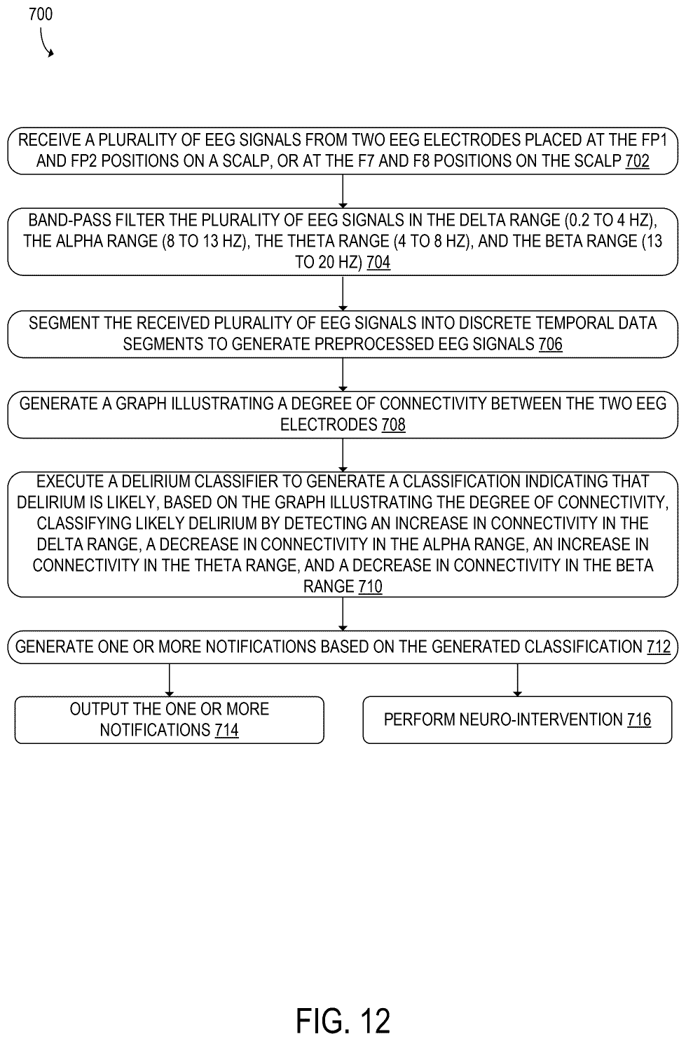

Referring to FIG. 12, a flow chart is illustrated of a sixth method 700 for real-time delirium classification. The sixth method 700 may be implemented on the delirium classification computing system 10 illustrated in FIG. 1A above, which includes the delirium classification computing device 12 and sensor system 26. Alternatively, other suitable computing hardware and software may be utilized.

At 702, the method includes receiving a plurality of EEG signals from two EEG electrodes placed at the Fp1 and Fp2 positions on a scalp, or at the F7 and F8 positions on the scalp. At 704, the method includes band-pass filtering the plurality of EEG signals in the delta range (0.1 to 4 Hz), the alpha range (8 to 12 Hz), the theta range (4 to 8 Hz), and the beta range (12 to 20 Hz). At 706, the method includes segmenting the received plurality of EEG signals into discrete temporal data segments to generate preprocessed EEG signals. At 708, the method includes generating a graph indicating a degree of functional connectivity between the two EEG electrodes. At 710, the method includes executing a delirium classifier to generate a classification indicating whether or not delirium is likely, based on the graph illustrating the degree of functional connectivity. Delirium is classified by detecting an increase in functional connectivity in the delta range, a decrease in functional connectivity in the alpha range, an increase in functional connectivity in a theta range, and a decrease in functional connectivity in a beta range. At 712, the method includes generating one or more notifications based on the generated classification. At 714, the method includes outputting the one or more notifications. The method may also include, at 716, performing a neuro-intervention based on the generated classification. The neuro-intervention may include localized or systemic delivery of therapeutic substances, ultrasonic treatment, visual stimulation, auditory stimulation, magnetic stimulation, and/or electrical stimulation.

It will be appreciated that, similarly to the second method 300, the third method 400, the fourth method 500, the fifth method 600, and the sixth method 700 may also generate a classification and a confidence score based on a weighted score and a probability score by receiving electronic health records and/or supplemental health data, and generating a probability score in addition to a classification, so that the outputted notifications may also include a confidence score, and a neuro-intervention may be performed when the confidence score is above a predetermined confidence score threshold.

The systems and methods described herein have the potential benefits of providing an objective, quantifiable way of classifying likely delirium in real-time, thereby reducing reliance on subjective clinical assessments. An earlier detection of delirium may be enabled, potentially leading to more timely and effective interventions. Only a single EEG electrode or pair of EEG electrodes are applied on the scalp or inside the ear for delirium monitoring, making this a convenient, non-invasive solution which is suitable for continuous monitoring in various clinical settings.

It will be appreciated that, in the above disclosure, the frequencies of EEG signals are generally described using commonly accepted frequency bands, including a delta range from approximately 0 to 4 Hz, a theta range from approximately 4 to 8 Hz, an alpha range from approximately 8 to 12 Hz, a beta range from approximately 13 to 20 Hz, a lower frequency range from 0 to 12 Hz, and a higher frequency range of 13 Hz or greater. However, in some implementations, the lower limit of the delta frequency range may be 0 Hz, 0.1 Hz, 0.2 Hz, or 1 Hz. Likewise, the lower limit of the beta frequency range may be 12 Hz or 13 Hz. The upper limit of the alpha frequency range may be 12 Hz or 13 Hz. The upper limit of the lower frequency range may alternatively be defined as 12 Hz or 13 Hz. The lower limit of the higher frequency range may be 12 Hz or 13 Hz. The upper limit of the higher frequency range may be 20 Hz, 50 Hz, or 100 Hz, depending on the implementation.

The above-described systems and methods may be applied in remote brain monitoring solutions, thereby extending the potential of this technology across various hospital environments, signifying a crucial step towards mitigating the adverse effects of diseases like COVID-19 or brain injury. Continuous monitoring and detection of delirium may help prevent neurocognitive decline, extend the reach of scarce healthcare expertise (such as neurologists or intensive care physicians), assist with brain health triage for in-hospital evaluation and admission, provide for remote diagnosis in a platform-agnostic manner, and facilitate earlier intervention to prevent failure of vital organs, namely the brain.

While the above-described systems and methods have been described with relation to the detection of the likelihood of delirium, it will be appreciated that the application of these systems and methods are not limited to this use case, as they may prove able to detect other types of brain failures. For example, the specific type of delirium (hypoactive delirium, hyperactive delirium, or mixed hypoactive and hyperactive delirium), the severity of delirium (sub-syndromal, mild, moderate, severe), or the underlying cause of the delirium may be identified by taking into account accelerometry data and vital signs in electronic health records or supplemental health data.

In some embodiments, the methods and processes described herein may be tied to a computing system of one or more computing devices. In particular, such methods and processes may be implemented as a computer-application program or service, an API, a library, and/or other computer-program product.

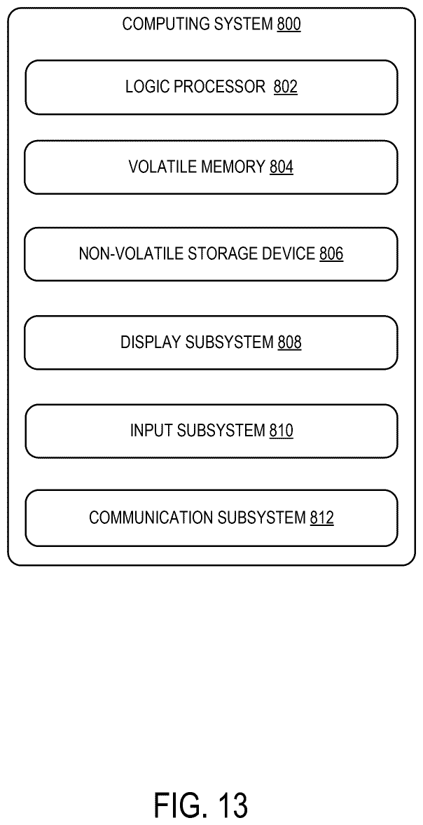

FIG. 13 schematically shows a non-limiting embodiment of a computing system 800 that can enact one or more of the methods and processes described above. Computing system 800 is shown in simplified form. Computing system 800 may embody the computing system 10 described above and illustrated in FIG. 1A or the computing system 100 described above and illustrated in FIG. 1B. Components of computing system 800 may be included in one or more personal computers, server computers, tablet computers, home-entertainment computers, network computing devices, video game devices, mobile computing devices, mobile communication devices (for example, smartphone), and/or other computing devices, and wearable computing devices such as smart wristwatches and head mounted augmented reality devices.

Computing system 800 includes processing circuitry 802, volatile memory 804, and a non-volatile storage device 806. Computing system 800 may optionally include a display subsystem 808, input subsystem 810, communication subsystem 812, and/or other components not shown in FIG. 13.

Processing circuitry typically includes one or more logic processors, which are physical devices configured to execute instructions. For example, the logic processors may be configured to execute instructions that are part of one or more applications, programs, routines, libraries, objects, components, data structures, or other logical constructs. Such instructions may be implemented to perform a task, implement a data type, transform the state of one or more components, achieve a technical effect, or otherwise arrive at a desired result.

The logic processor may include one or more physical processors configured to execute software instructions. Additionally or alternatively, the logic processor may include one or more hardware logic circuits or firmware devices configured to execute hardware-implemented logic or firmware instructions. Processors of the processing circuitry 802 may be single-core or multi-core, and the instructions executed thereon may be configured for sequential, parallel, and/or distributed processing. Individual components of the processing circuitry optionally may be distributed among two or more separate devices, which may be remotely located and/or configured for coordinated processing. For example, aspects of the computing system disclosed herein may be virtualized and executed by remotely accessible, networked computing devices configured in a cloud-computing configuration. In such a case, these virtualized aspects are run on different physical logic processors of various different machines, it will be understood. These different physical logic processors of the different machines will be understood to be collectively encompassed by processing circuitry 802.

Non-volatile storage device 806 includes one or more physical devices configured to hold instructions executable by the processing circuitry to implement the methods and processes described herein. When such methods and processes are implemented, the state of non-volatile storage device 806 may be transformed—e.g., to hold different data.

Non-volatile storage device 806 may include physical devices that are removable and/or built in. Non-volatile storage device 806 may include optical memory, semiconductor memory, and/or magnetic memory, or other mass storage device technology. Non-volatile storage device 806 may include nonvolatile, dynamic, static, read/write, read-only, sequential-access, location-addressable, file-addressable, and/or content-addressable devices. It will be appreciated that non-volatile storage device 806 is configured to hold instructions even when power is cut to the non-volatile storage device 806.

Volatile memory 804 may include physical devices that include random access memory. Volatile memory 804 is typically utilized by processing circuitry 802 to temporarily store information during processing of software instructions. It will be appreciated that volatile memory 804 typically does not continue to store instructions when power is cut to the volatile memory 804.

Aspects of processing circuitry 802, volatile memory 804, and non-volatile storage device 806 may be integrated together into one or more hardware-logic components. Such hardware-logic components may include field-programmable gate arrays (FPGAs), program- and application-specific integrated circuits (PASIC/ASICs), program- and application-specific standard products (PSSP/ASSPs), SOC, and complex programmable logic devices (CPLDs), for example.

The terms “module,” “program,” and “engine” may be used to describe an aspect of computing system 800 typically implemented in software by a processor to perform a particular function using portions of volatile memory, which function involves transformative processing that specially configures the processor to perform the function. Thus, a module, program, or engine may be instantiated via processing circuitry 802 executing instructions held by non-volatile storage device 806, using portions of volatile memory 804. It will be understood that different modules, programs, and/or engines may be instantiated from the same application, service, code block, object, library, routine, API, function, etc. Likewise, the same module, program, and/or engine may be instantiated by different applications, services, code blocks, objects, routines, APIs, functions, etc. The terms “module,” “program,” and “engine” may encompass individual or groups of executable files, data files, libraries, drivers, scripts, database records, etc.

When included display subsystem 808 may be used to present a visual representation of data held by non-volatile storage device 806. The visual representation may take the form of a GUI. As the herein described methods and processes change the data held by the non-volatile storage device, and thus transform the state of the non-volatile storage device, the state of display subsystem 808 may likewise be transformed to visually represent changes in the underlying data. Display subsystem 808 may include one or more display devices utilizing virtually any type of technology. Such display devices may be combined with processing circuitry 802, volatile memory 804, and/or non-volatile storage device 806 in a shared enclosure, or such display devices may be peripheral display devices.

When included, input subsystem 810 may comprise or interface with one or more user-input devices such as a keyboard, mouse, touch screen, camera, or microphone.

When included, communication subsystem 812 may be configured to communicatively couple various computing devices described herein with each other, and with other devices. Communication subsystem 812 may include wired and/or wireless communication devices compatible with one or more different communication protocols. As non-limiting examples, the communication subsystem may be configured for communication via a wired or wireless local-or wide-area network, broadband cellular network, etc. In some embodiments, the communication subsystem may allow computing system 800 to send and/or receive messages to and/or from other devices via a network such as the Internet.

The following paragraphs provide additional support for the claims of the subject application. In one aspect, a delirium detection computing system is provided, comprising a sensor system comprising one or more electroencephalography (EEG) electrodes configured to generate a plurality of EEG signals, processing circuitry, and a non-volatile memory storing executable instructions that, in response to execution by the processing circuitry, cause the processing circuitry to receive the plurality of EEG signals, preprocess the received plurality of EEG signals to generate preprocessed EEG signals, extract features to generate EEG representations based on the preprocessed EEG signals, execute a delirium classifier to generate a classification indicating whether or not delirium is likely, based on the generated EEG representations, and generate and output one or more notifications based on the generated classification. In this aspect, additionally or alternatively, the delirium classifier is further configured to generate a probability score indicating a degree of certainty about the classification, and the one or more notifications are generated and outputted based on the generated classification and the probability score. In this aspect, additionally or alternatively, the processing circuitry is further configured to receive health data, generate a weighted score based on the received health data, generate a confidence score based on the weighted score and the probability score, and generate and output the one or more notifications based on the generated classification and the confidence score. In this aspect, additionally or alternatively, the one or more notifications include at least one of an audio notification with musical pitches or sound frequencies indicating the confidence score or a visual notification with colors indicating the confidence score. In this aspect, additionally or alternatively, the processing circuitry is further configured to generate a severity score based on the weighted score, and generate and output the one or more notifications based on the generated classification, the severity score, and the confidence score. In this aspect, additionally or alternatively, the one or more notifications include at least one of an audio notification with musical pitches or sound frequencies indicating the severity score or a visual notification with colors indicating the severity score. In this aspect, additionally or alternatively, the system further comprises an intervention device, and the processing circuitry is further configured to determine whether the confidence score is above a predetermined confidence score threshold, and responsive to determining that the confidence score is above the predetermined confidence score threshold, perform a neuro-intervention via the intervention device. In this aspect, additionally or alternatively, the neuro-intervention is at least one of a delivery of therapeutic substances, ultrasonic treatment, visual stimulation, auditory stimulation, magnetic stimulation, or electrical stimulation. In this aspect, additionally or alternatively, the delirium classifier is configured to generate the classification that delirium is likely by detecting power spectra in the EEG signals according to predetermined criteria, and the predetermined criteria include at least one of a first condition of increased power in a delta range, a second condition of increased power in a theta range, a third condition of decreased ratio of a power in an alpha range to a power in the delta range, a fourth condition of decreased ratio of power in the alpha range to power in the theta range, a fifth condition of decreased ratio of power in a beta range to power in the delta range, a sixth condition of decreased ratio of power in the beta range to power in the theta range, or a seventh condition of increased ratio of power in the delta plus theta plus alpha frequency range to power in the beta plus low gamma frequency range. In this aspect, additionally or alternatively, the EEG representations are attractors generated from time-delayed embeddings created from the preprocessed EEG signals, and the delirium classifier generates the classification based on changes in an ellipse radius ratio of the attractors. In this aspect, additionally or alternatively, the EEG representations are generated based on a change in relative entropy calculated from the preprocessed EEG signals, and the delirium classifier generates the classification based on the change in entropy over time. In this aspect, additionally or alternatively, the sensor system comprises two EEG electrodes configured to be placed on bilateral locations on a scalp, the EEG representations are graphs illustrating a degree of synchrony or functional connectivity between the two EEG electrodes, and the delirium classifier generates the classification based on a change in the synchrony or functional connectivity over time. In this aspect, additionally or alternatively, the delirium classifier is configured to generate the classification that delirium is likely by detecting an increase in functional connectivity in a delta range, a decrease in functional connectivity in an alpha range, an increase in functional connectivity in a theta range, and a decrease in functional connectivity in a beta range. In this aspect, additionally or alternatively, the functional connectivity is measured based on changes in a weighted Phase Lag Index. In this aspect, additionally or alternatively, the two EEG electrodes are placed at Fp1 and Fp2 positions on the scalp, or at F7 and F8 positions on the scalp. In this aspect, additionally or alternatively, the functional connectivity is quantified as a renormalized partial directed coherence. In this aspect, additionally or alternatively, the renormalized partial directed coherence quantifies a frequency and a strength of functional connectivity for one of a frontal to frontotemporal direction, frontotemporal to frontal direction, frontal to central direction, or central to frontal direction. In this aspect, additionally or alternatively, the delirium classifier includes a time-continuous machine learning model trained to differentiate between delirious states and non-delirious states in the EEG representations. In this aspect, additionally or alternatively, the time-continuous machine learning model is a liquid time-constant network (LTCN).

In another aspect, a delirium detection computing method is provided, comprising receiving a plurality of electroencephalography (EEG) signals from one or more EEG electrodes, preprocessing the received plurality of EEG signals to generate preprocessed EEG signals, extracting features to generate EEG representations based on the preprocessed EEG signals, executing a delirium classifier to generate a classification indicating whether or not delirium is likely, based on the generated EEG representations, and generating and outputting one or more notifications based on the generated classification. In this aspect, additionally or alternatively, the delirium classifier is executed to further generate a probability score indicating a degree of certainty about the classification, and the one or more notifications are generated and outputted based on the generated classification and the probability score. In this aspect, additionally or alternatively, the method further comprises receiving health data, generating a weighted score based on the received health data, generating a confidence score based on the weighted score and the probability score, and generating and outputting the one or more notifications based on the generated classification and the confidence score. In this aspect, additionally or alternatively, the one or more notifications include at least one of an audio notification with musical pitches or sound frequencies indicating the confidence score or a visual notification with colors indicating the confidence score. In this aspect, additionally or alternatively, the method further comprises generating a severity score based on the weighted score, and generating and outputting the one or more notifications based on the generated classification, the severity score, and the confidence score. In this aspect, additionally or alternatively, the one or more notifications include at least one of an audio notification with musical pitches or sound frequencies indicating the severity score or a visual notification with colors indicating the severity score. In this aspect, additionally or alternatively, the method further comprises determining whether the confidence score is above a predetermined confidence score threshold, and responsive to determining that the confidence score is above the predetermined confidence score threshold, performing a neuro-intervention via an intervention device. In this aspect, additionally or alternatively, the neuro-intervention is at least one of a delivery of therapeutic substances, ultrasonic treatment, visual stimulation, auditory stimulation, magnetic stimulation, or electrical stimulation. In this aspect, additionally or alternatively, the classification that delirium is likely is generated by detecting power spectra in the EEG signals according to predetermined criteria, and the predetermined criteria include at least one of a first condition of increased power in a delta range, a second condition of increased power in a theta range, a third condition of decreased ratio of a power in an alpha range to a power in the delta range, a fourth condition of decreased ratio of power in the alpha range to power in the theta range, a fifth condition of decreased ratio of power in a beta range to power in the delta range, a sixth condition of decreased ratio of power in the beta range to power in the theta range, or a seventh condition of increased ratio of power in the delta plus theta plus alpha frequency range to power in the beta plus low gamma frequency range. In this aspect, additionally or alternatively, the EEG representations are attractors generated from time-delayed embeddings created from the preprocessed EEG signals, and the classification is generated based on changes in an ellipse radius ratio of the attractors. In this aspect, additionally or alternatively, the EEG representations are generated based on a change in relative entropy calculated from the preprocessed EEG signals, and the classification is generated based on the change in entropy over time. In this aspect, additionally or alternatively, the EEG representations are graphs illustrating a degree of synchrony or functional connectivity between two EEG electrodes, and the delirium classifier generates the classification based on a change in the synchrony or functional connectivity over time. In this aspect, additionally or alternatively, the classification that delirium is likely is generated by detecting an increase in functional connectivity in a delta range, a decrease in functional connectivity in an alpha range, an increase in functional connectivity in a theta range, and a decrease in functional connectivity in a beta range. In this aspect, additionally or alternatively, the functional connectivity is measured based on changes in a weighted Phase Lag Index. In this aspect, additionally or alternatively, the two EEG electrodes are placed at Fp1 and Fp2 positions on a scalp, or at F7 and F8 positions on the scalp. In this aspect, additionally or alternatively, the functional connectivity is quantified as a renormalized partial directed coherence. In this aspect, additionally or alternatively, the renormalized partial directed coherence quantifies a frequency and a strength of functional connectivity for one of a frontal to frontotemporal direction, frontotemporal to frontal direction, frontal to central direction, or central to frontal direction.

In another aspect, a delirium detection computing device is provided, comprising one or more electroencephalography (EEG) electrodes configured to generate a plurality of EEG signals at a position in a forehead region or a periauricular region on a scalp or at an in-ear position, processing circuitry, and a non-volatile memory storing executable instructions that, in response to execution by the processing circuitry, cause the processing circuitry to receive the plurality of EEG signals, segment the received plurality of EEG signals into discrete temporal data segments to generate preprocessed EEG signals, extract features to generate EEG graphs based on the preprocessed EEG signals, wherein to generate each EEG graph, a current EEG graph is superimposed onto a preceding EEG graph from a time-delayed segment, execute a delirium classifier to generate a classification indicating whether or not delirium is likely, based on the generated EEG graphs, and generate and output one or more notifications based on the generated classification, wherein the delirium classifier is configured as a time-continuous machine learning model. In this aspect, additionally or alternatively, the time-continuous machine learning model is a liquid time-constant network (LTCN).

In another aspect, a brain state detection computing system is provided, comprising a sensor system comprising one or more electroencephalography (EEG) electrodes configured to generate a plurality of EEG signals, processing circuitry, and a non-volatile memory storing executable instructions that, in response to execution by the processing circuitry, cause the processing circuitry to receive the plurality of EEG signals, preprocess the received plurality of EEG signals to generate preprocessed EEG signals, extract features to generate EEG representations based on the preprocessed EEG signals, execute a brain state classifier to generate a classification of a predetermined brain state, based on the generated EEG representations, and generate and output one or more notifications based on the generated classification. In this aspect, additionally or alternatively, the predetermined brain state is at least one of a neurocognitive disorder, neurological dysfunction, a neurological impairment, sedation, oversedation, confusion, encephalopathy, stroke, brain failure, or general brain health degradation.

“And/or” as used herein is defined as the inclusive or V, as specified by the following truth table:

| A | B | A ∨ B |
|---|---|---|
| True | True | True |
| True | False | True |
| False | True | True |
| False | False | False |

It will be understood that the configurations and/or approaches described herein are exemplary in nature, and that these specific embodiments or examples are not to be considered in a limiting sense, because numerous variations are possible. The specific routines or methods described herein may represent one or more of any number of processing strategies. As such, various acts illustrated and/or described may be performed in the sequence illustrated and/or described, in other sequences, in parallel, or omitted. Likewise, the order of the above-described processes may be changed.

The subject matter of the present disclosure includes all novel and non-obvious combinations and sub-combinations of the various processes, systems and configurations, and other features, functions, acts, and/or properties disclosed herein, as well as any and all equivalents thereof.

1. A delirium detection computing system, comprising:
a sensor system comprising one or more electroencephalography (EEG) electrodes configured to generate a plurality of EEG signals;
processing circuitry; and
a non-volatile memory storing executable instructions that, in response to execution by the processing circuitry, cause the processing circuitry to:
receive the plurality of EEG signals;
preprocess the received plurality of EEG signals to generate preprocessed EEG signals;
extract features to generate EEG representations based on the preprocessed EEG signals;
execute a delirium classifier to generate a classification indicating whether or not delirium is likely, based on the generated EEG representations; and
generate and output one or more notifications based on the generated classification.

2. The delirium detection computing system of claim 1, wherein
the delirium classifier is further configured to generate a probability score indicating a degree of certainty about the classification; and
the one or more notifications are generated and outputted based on the generated classification and the probability score.

3. The delirium detection computing system of claim 2, wherein the processing circuitry is further configured to:
receive health data;
generate a weighted score based on the received health data;
generate a confidence score based on the weighted score and the probability score; and
generate and output the one or more notifications based on the generated classification and the confidence score.

4. The delirium detection computing system of claim 3, wherein the one or more notifications include at least one of an audio notification with musical pitches or sound frequencies indicating the confidence score or a visual notification with colors indicating the confidence score.

5. The delirium detection computing system of claim 3, wherein the processing circuitry is further configured to:
generate a severity score based on the weighted score; and
generate and output the one or more notifications based on the generated classification, the severity score, and the confidence score.

6. The delirium detection computing system of claim 5, wherein the one or more notifications include at least one of an audio notification with musical pitches or sound frequencies indicating the severity score or a visual notification with colors indicating the severity score.

7. The delirium detection computing system of claim 3, further comprising an intervention device, wherein the processing circuitry is further configured to:
determine whether the confidence score is above a predetermined confidence score threshold; and
responsive to determining that the confidence score is above the predetermined confidence score threshold, perform a neuro-intervention via the intervention device.

8. The delirium detection computing system of claim 7, wherein the neuro-intervention is at least one of a delivery of therapeutic substances, ultrasonic treatment, visual stimulation, auditory stimulation, magnetic stimulation, or electrical stimulation.

9. The delirium detection computing system of claim 1, wherein
the delirium classifier is configured to generate the classification that delirium is likely by detecting power spectra in the EEG signals according to predetermined criteria; and
the predetermined criteria include at least one of a first condition of increased power in a delta range; a second condition of increased power in a theta range; a third condition of decreased ratio of a power in an alpha range to a power in the delta range; a fourth condition of decreased ratio of power in the alpha range to power in the theta range; a fifth condition of decreased ratio of power in a beta range to power in the delta range; a sixth condition of decreased ratio of power in the beta range to power in the theta range; or a seventh condition of increased ratio of power in the delta plus theta plus alpha frequency range to power in the beta plus low gamma frequency range.

10. The delirium detection computing system of claim 1, wherein
the EEG representations are attractors generated from time-delayed embeddings created from the preprocessed EEG signals; and
the delirium classifier generates the classification based on changes in an ellipse radius ratio of the attractors.

11. The delirium detection computing system of claim 1, wherein
the EEG representations are generated based on a change in relative entropy calculated from the preprocessed EEG signals; and
the delirium classifier generates the classification based on the change in entropy over time.

12. The delirium detection computing system of claim 1, wherein
the sensor system comprises two EEG electrodes configured to be placed on bilateral locations on a scalp;
the EEG representations are graphs illustrating a degree of synchrony or functional connectivity between the two EEG electrodes; and
the delirium classifier generates the classification based on a change in the synchrony or functional connectivity over time.

13. The delirium detection computing system of claim 12, wherein the delirium classifier is configured to generate the classification that delirium is likely by detecting an increase in functional connectivity in a delta range, a decrease in functional connectivity in an alpha range, an increase in functional connectivity in a theta range, and a decrease in functional connectivity in a beta range.

14. The delirium detection computing system of claim 13, wherein the functional connectivity is measured based on changes in a weighted Phase Lag Index.

15. The delirium detection computing system of claim 12, wherein the two EEG electrodes are placed at Fp1 and Fp2 positions on the scalp, or at F7 and F8 positions on the scalp.

16. The delirium detection computing system of claim 12, wherein the functional connectivity is quantified as a renormalized partial directed coherence.

17. The delirium detection computing system of claim 16, wherein the renormalized partial directed coherence quantifies a frequency and a strength of functional connectivity for one of a frontal to frontotemporal direction, frontotemporal to frontal direction, frontal to central direction, or central to frontal direction.

18. The delirium detection computing system of claim 1, wherein the delirium classifier includes a time-continuous machine learning model trained to differentiate between delirious states and non-delirious states in the EEG representations.

19. The delirium detection computing system of claim 18, wherein the time-continuous machine learning model is a liquid time-constant network (LTCN).

20. A delirium detection computing method, comprising:
receiving a plurality of electroencephalography (EEG) signals from one or more EEG electrodes;
preprocessing the received plurality of EEG signals to generate preprocessed EEG signals;
extracting features to generate EEG representations based on the preprocessed EEG signals;
executing a delirium classifier to generate a classification indicating whether or not delirium is likely, based on the generated EEG representations; and
generating and outputting one or more notifications based on the generated classification.

21. The delirium detection computing method of claim 20, wherein
the delirium classifier is executed to further generate a probability score indicating a degree of certainty about the classification; and
the one or more notifications are generated and outputted based on the generated classification and the probability score.

22. The delirium detection computing method of claim 21, further comprising:
receiving health data;
generating a weighted score based on the received health data;
generating a confidence score based on the weighted score and the probability score; and
generating and outputting the one or more notifications based on the generated classification and the confidence score.

23. The delirium detection computing method of claim 22, wherein the one or more notifications include at least one of an audio notification with musical pitches or sound frequencies indicating the confidence score or a visual notification with colors indicating the confidence score.

24. The delirium detection computing method of claim 22, further comprising:
generating a severity score based on the weighted score; and
generating and outputting the one or more notifications based on the generated classification, the severity score, and the confidence score.

25. The delirium detection computing method of claim 24, wherein the one or more notifications include at least one of an audio notification with musical pitches or sound frequencies indicating the severity score or a visual notification with colors indicating the severity score.

26. The delirium detection computing method of claim 22, further comprising:
determining whether the confidence score is above a predetermined confidence score threshold; and
responsive to determining that the confidence score is above the predetermined confidence score threshold, performing a neuro-intervention via an intervention device.

27. The delirium detection computing method of claim 26, wherein the neuro-intervention is at least one of a delivery of therapeutic substances, ultrasonic treatment, visual stimulation, auditory stimulation, magnetic stimulation, or electrical stimulation.

28. The delirium detection computing method of claim 20, wherein
the classification that delirium is likely is generated by detecting power spectra in the EEG signals according to predetermined criteria; and
the predetermined criteria include at least one of a first condition of increased power in a delta range; a second condition of increased power in a theta range; a third condition of decreased ratio of a power in an alpha range to a power in the delta range; a fourth condition of decreased ratio of power in the alpha range to power in the theta range; a fifth condition of decreased ratio of power in a beta range to power in the delta range; a sixth condition of decreased ratio of power in the beta range to power in the theta range; or a seventh condition of increased ratio of power in the delta plus theta plus alpha frequency range to power in the beta plus low gamma frequency range.

29. The delirium detection computing method of claim 20, wherein
the EEG representations are attractors generated from time-delayed embeddings created from the preprocessed EEG signals; and
the classification is generated based on changes in an ellipse radius ratio of the attractors.

30. The delirium detection computing method of claim 20, wherein
the EEG representations are generated based on a change in relative entropy calculated from the preprocessed EEG signals; and
the classification is generated based on the change in entropy over time.

31. The delirium detection computing method of claim 20, wherein
the EEG representations are graphs illustrating a degree of synchrony or functional connectivity between two EEG electrodes; and
the delirium classifier generates the classification based on a change in the synchrony or functional connectivity over time.

32. The delirium detection computing method of claim 31, wherein the classification that delirium is likely is generated by detecting an increase in functional connectivity in a delta range, a decrease in functional connectivity in an alpha range, an increase in functional connectivity in a theta range, and a decrease in functional connectivity in a beta range.

33. The delirium detection computing method of claim 32, wherein the functional connectivity is measured based on changes in a weighted Phase Lag Index.

34. The delirium detection computing method of claim 31, wherein the two EEG electrodes are placed at Fp1 and Fp2 positions on a scalp, or at F7 and F8 positions on the scalp.

35. The delirium detection computing method of claim 31, wherein the functional connectivity is quantified as a renormalized partial directed coherence.

36. The delirium detection computing method of claim 35, wherein the renormalized partial directed coherence quantifies a frequency and a strength of functional connectivity for one of a frontal to frontotemporal direction, frontotemporal to frontal direction, frontal to central direction, or central to frontal direction.

37. A delirium detection computing device, comprising:
one or more electroencephalography (EEG) electrodes configured to generate a plurality of EEG signals at a position in a forehead region or a periauricular region on a scalp or at an in-ear position;
processing circuitry; and
a non-volatile memory storing executable instructions that, in response to execution by the processing circuitry, cause the processing circuitry to:
receive the plurality of EEG signals;
segment the received plurality of EEG signals into discrete temporal data segments to generate preprocessed EEG signals;
extract features to generate EEG graphs based on the preprocessed EEG signals, wherein to generate each EEG graph, a current EEG graph is superimposed onto a preceding EEG graph from a time-delayed segment;
execute a delirium classifier to generate a classification indicating whether or not delirium is likely, based on the generated EEG graphs; and
generate and output one or more notifications based on the generated classification, wherein
the delirium classifier is configured as a time-continuous machine learning model.

38. The delirium detection computing device of claim 37, wherein the time-continuous machine learning model is a liquid time-constant network (LTCN).

39. A brain state detection computing system, comprising:
a sensor system comprising one or more electroencephalography (EEG) electrodes configured to generate a plurality of EEG signals;
processing circuitry; and
a non-volatile memory storing executable instructions that, in response to execution by the processing circuitry, cause the processing circuitry to:
receive the plurality of EEG signals;
preprocess the received plurality of EEG signals to generate preprocessed EEG signals;
extract features to generate EEG representations based on the preprocessed EEG signals;
execute a brain state classifier to generate a classification of a predetermined brain state, based on the generated EEG representations; and
generate and output one or more notifications based on the generated classification.

40. The brain state detection computing system of claim 39, wherein the predetermined brain state is at least one of a neurocognitive disorder, neurological dysfunction, a neurological impairment, sedation, oversedation, confusion, encephalopathy, stroke, brain failure, or general brain health degradation.
